(19) United States
(12) Patent Application Publication
(10) Pub. No.: US 2018/0104380 A1
(43) Pub. Date: Apr. 19, 2018

(54) CARBON PARTICULATES AND COMPOSITES THEREOF FOR MUSCULOSKELETAL AND SOFT TISSUE REGENERATION

(71) Applicant: Wichita State University, Wichita, KS (US)

(72) Inventors: Stephen Miller, Wichita, KS (US); Nora Strong, Park City, KS (US); Joel White, Wichita, KS (US)

(21) Appl. No.: 15/561,150

(22) PCT Filed: Mar. 23, 2016
(86) PCT No.: PCT/US2016/023745

§ 371 Date: Sep. 25, 2017

Related U.S. Application Data
(60) Provisional application No. 62/138,133, filed on Mar. 25, 2015.

(51) Int. Cl. A61L 27/08; A61L 27/44; A61L 27/56; A61L 27/54

(52) U.S. Cl. CPC A61L 27/08; A61L 27/443; A61L 2430/02; A61L 27/54; A61L 27/56

(57) ABSTRACT

The present application draws attention to particulate materials derived from carbonaceous materials, such as carbon foam, as a platform technology for multiple medical procedures performed to address diverse tissue deficits and/or pathologies. The particulates are biocompatible, permissive of cell attachment and differentiation, and provide a 3-dimensional substrate that is permissive of neo-vascularization. Methods are disclosed herein whereby the application and subsequent bodily interactions with the particulate materials with or without composite materials are optimized to provide a technological platform useful for multiple regenerative procedures.

### CROSS-REFERENCE TO RELATED APPLICATIONS

The present application claims the priority benefit of U.S. Provisional Patent Application Ser. No. 62/138,133, filed Mar. 25, 2015, entitled PARTICULATE VITREOUS CARBON FOAM AND COMPOSITIONS THEREOF FOR MUSCULOSKELETAL AND SOFT TISSUE REGENERATION, incorporated by reference in its entirety herein.

### BACKGROUND

Regenerative medicine, in which therapeutics, devices and repair cells are applied to help the body heal itself, is currently one of the fastest growing areas of medical practice. The clinical impact of these new initiatives however has been constrained owing to a lack of materials and suitable geometries that fulfill the mandates of safety, efficacy, ease of use and cost-effectiveness.

Bone is a regenerative tissue that can sometimes effectively repair fracture damage and small voids, but trauma that results in the loss of a large bone segment requires the use of a bone void filler to bridge the gap between bone segments. Bone void fillers are natural or synthetic materials placed into the bone defect to assist in bone regeneration and provide a three-dimensional structure to which bone tissue and cells can attach or be transplanted to regrow and repair damaged bone segments. Autologous, allogeneic, or xenogeneic bone grafts can be used for this purpose. However, in major traumatic injury, there is usually insufficient autologous bone available as avoid filler. In addition, harvesting of the graft from the donor site for bone autografts causes secondary trauma to the patient. This is avoided by the use of allografts and xenografts, however, these bone grafts can transmit pathogens, cause adverse immunological responses, and their efficacy is not supported by level I clinical evidence.

Allogeneic and synthetic bone void fillers such as demineralized bone matrix, ceramic granules or polymethyl methacrylate have also been employed clinically. However these bone void fillers are generally intended for use only in voids that are not intrinsic to the stability of the bone structure, since most commercially-available materials possess insufficient strength to resist the compressive or tensile forces acting in a particular anatomic environment. Implants used for bone voids intrinsic to structural stability of the bone often involve permanent implant materials formed from metal, polymers, or composites. These types of implants suffer from their propensity to corrode or leach. In addition, all metals used as orthopedic implants, most ceramics, and many polymers are not bioresorbable, and cannot be replaced by the patient's own bone tissue. Thus, there remains a need in the art for a temporary bone void filler that provides a template for bone remodeling and osteogenic signals that also gradually degrades and is resorbed as it is replaced by bone tissue until bone generation/regeneration is complete.

The practice of orthopedic medicine is at present substantially hindered by the lack of suitable bone grafting materials that meet these stringent criteria. Among these limiting factors are harvest site morbidities (for autologous bone grafts), paucity of suitable donors and health issue (allografts), lack of substantial clinical evidence for induction of new bone (synthetic glass and ceramic materials), and recent recognition of serious off-target effects of the current leading bone graft substitute that uses delivery of potent cytokines (BMP-2) on a collagen sponge (InFuse, Medtronic, Inc.).

### SUMMARY OF THE INVENTION

Applicants have previously conducted extensive proof-of-concept work on bone regeneration using monolithic, porous vitreous carbon implant materials as described in US 2012/0185047 and US 2012/0095558, incorporated by reference herein. There remain unmet needs for bone grafting procedures that lie outside the unique clinical demands of the repair of long bone defects and spinal fusion. These include specialized procedures such as vertebral fracture stabilization, repair of recalcitrant non-unions, oral-maxillofacial and dental repair, restoration of iliac crest harvest sites, restoration of long bone anatomy following extensive resection following tumor removal, as well as for soft tissue repair. Significant effort has been expended in the present work to expand the range of clinical applications to which vitreous carbon foam (VCF) materials can be put, with emphasis upon orthopedic applications, but equally as applicable to soft tissue regeneration including, vascular, epidermal, hepatic and cardiac tissues as well.

The present invention is broadly concerned with compositions for tissue and/or bone regeneration, which comprises a plurality of carbonaceous particulates and at least one osteopromotive and/or therapeutic agent. The carbonaceous particulates have a maximum surface-to-surface dimension of less than about 1,000 microns, and in some embodiments are non-porous (no macro-porous structure) granular bodies derived from carbon foam. This means that the carbon foam granules may have intrinsic microporosity (10-20 um, down to nanometer scale), but the macro-porous structure is no longer intact. In some embodiments, the particulates are dispersed in a carrier matrix. The at least one osteopromotive and/or therapeutic agent can be individually dispersed in the carrier matrix, but may also/alternatively be present in the composition as part of a coating on the carbonaceous particulates.

Also described herein are implants for tissue and/or bone regeneration. In one aspect, the implant can comprise a composition according to the various embodiments disclosed herein and a biocompatible mesh container that encloses the composition. In one aspect, the implant can comprise a composition according to the various embodiments disclosed herein and a biocompatible scaffold (e.g., collagen sponge, polymer sheet, etc.) in which the composition is embedded.

Methods are also described herein for tissue and/or bone regeneration in a subject having a need therefor. The methods generally comprise providing a composition according to the various embodiments disclosed herein, and implanting the composition into the subject at an implantation site having a bone defect or tissue damage. As part of the implanting the composition can be molded or plied into the desired shape to fit the contours of the implantation site and provide a precise interface with adjacent healthy bone or tissue. Implanting can also be accomplished by dispensing the composition from a syringe targeted to the site of implantation.

Kits for tissue and/or bone regeneration in a subject having a need therefor are also described. The kits generally comprise a composition according to the various embodiments disclosed herein, and instructions for implantation thereof in the subject at an implantation site having a bone defect or tissue damage. Appropriate tools can also be included in the kit, including a syringe for dispensing the composition (which may optionally be preloaded with the composition), tools for shaping and molding the composition, fixation devices for securing the composition/implant at the site of implantation, and the like.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Figure (FIG. 1A is an SEM image of reticulated vitreous carbon (RVC) foam annotated to illustrate nodes, ligaments, and cells that make up the matrix microstructure;

FIG. 1B is an SEM image of 45 ppi RVC that has been fragmented to break the ligaments and notes to create carbon-foam derived particulates;

FIG. 1C is an SEM image of 80 ppi RVC that has been fragmented to break the ligaments and notes to create carbon-foam derived particulates at ×25 magnification;

FIG. 1D is an SEM image of 80 ppi RVC that has been fragmented to break the ligaments and notes to create carbon-foam derived particulates at ×50 magnification;

FIG. 1E is an SEM image of 80 ppi RVC that has been fragmented to break the ligaments and notes to create carbon-foam derived particulates at ×75 magnification;

FIG. 1F is an SEM image of 100 ppi RVC that has been fragmented to break the ligaments and notes to create carbon-foam derived particulates at ×23 magnification;

FIG. 1G is an SEM image of 100 ppi RVC that has been fragmented to break the ligaments and notes to create carbon-foam derived particulates at ×50 magnification;

FIG. 1H is an SEM image of 100 ppi RVC that has been fragmented to break the ligaments and notes to create carbon-foam derived particulates at ×200 magnification;

FIG. 2 is a graph of a BMP-2 adsorption assay using DUOCEL® carbon foam;

FIG. 3 is graph demonstrating bioactivity of BMP-2 adsorbed on the carbon foam;

FIG. 4 is a graph showing the results from the alkaline phosphatase activity experiments in mouse myoblasts;

FIG. 5 is a graph showing the results from the alkaline phosphatase activity experiments in human osteoblasts;

FIG. 6 is a graph of the ELISA test results for the binding assay of BMP-2;

FIG. 7 is a graph of the ELISA test results for the binding assay of BMP-2 after additional rinsing with PBS;

FIG. 8 is a graph of the results of the MTT assay to determine replication of the mouse myoblasts, where the y-axis values are the relative MTT Metabolism (normalized to day 1, control=1);

FIG. 9 is a graph of the alkaline phosphatase specific activity with induction of BMP bound to the carbon foam;

FIG. 10 is a flowchart of the study protocol for Example 3;

FIG. 11A is a graph of the results of the protein release test for albumin;

FIG. 11B is a graph of the results of the protein release test for BMP-2;

FIG. 11C is a graph of the results of the protein release test for collagen-1;

FIG. 12 shows images of immunocytofluorescence analysis of induced osteoblasts;

FIG. 13 shows the results of culturing cells on carbon foam treated with albumin, BMP-2, collagen-1, and water;

FIG. 14 is a graph demonstrating BMP-2 binding to carbon foam particulates using alkaline phosphatase induction in C2C12 mouse myoblast cells; and

FIG. 15 shows images of ectopic bone formation induced by carbon foam particulates or carbon foam particulates coated with BMP-2 implanted into an abdominal muscle pouch of a Lewis rat for 30 days as measured using microCT.

### DETAILED DESCRIPTION OF PREFERRED EMBODIMENTS

The following sets forth the invention with reference to the preferred embodiments which may be illustrated in the Figures. It is to be understood, however, that these preferred embodiments are provided by way of example and nothing therein should be taken as a limitation upon the overall scope of the invention that is claimed. The various embodiments depicted in the Figures and described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

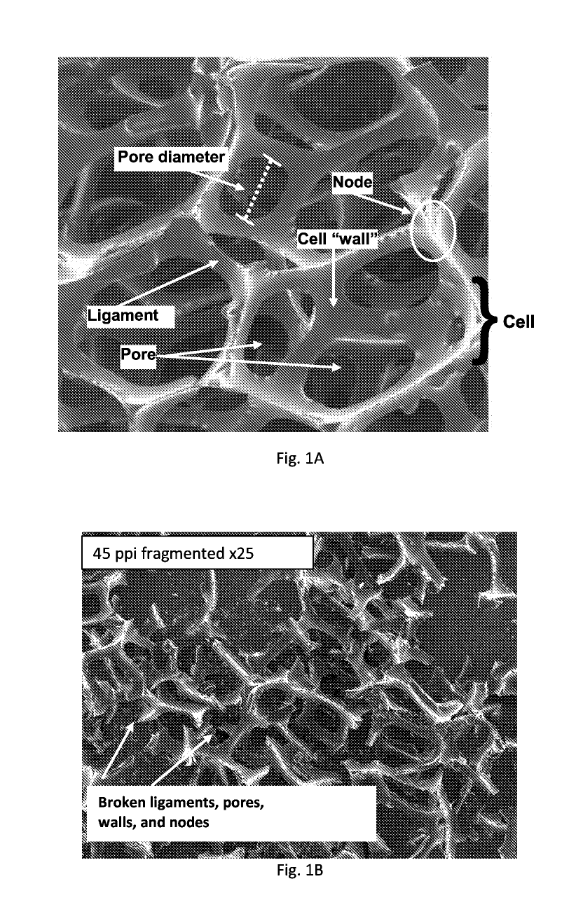

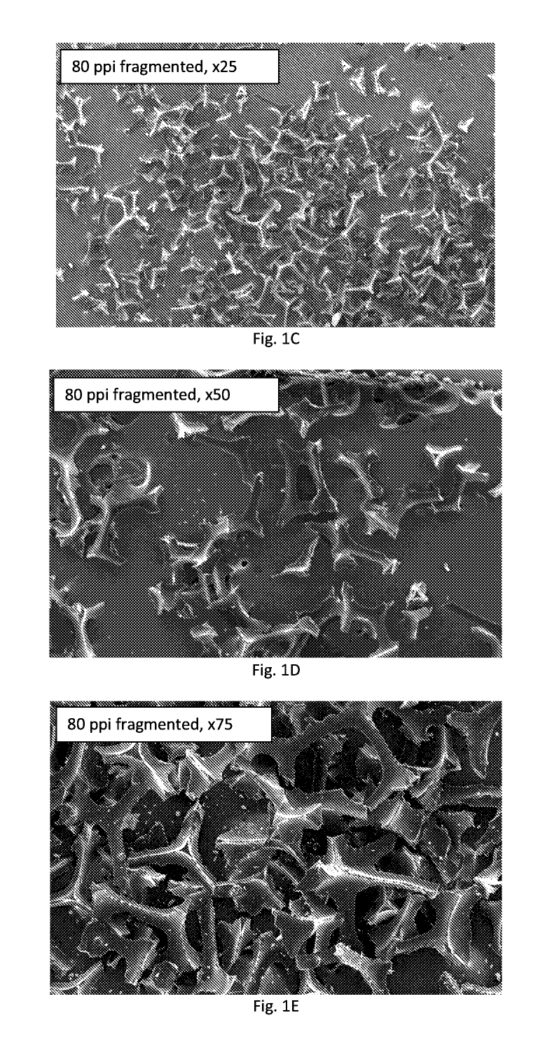

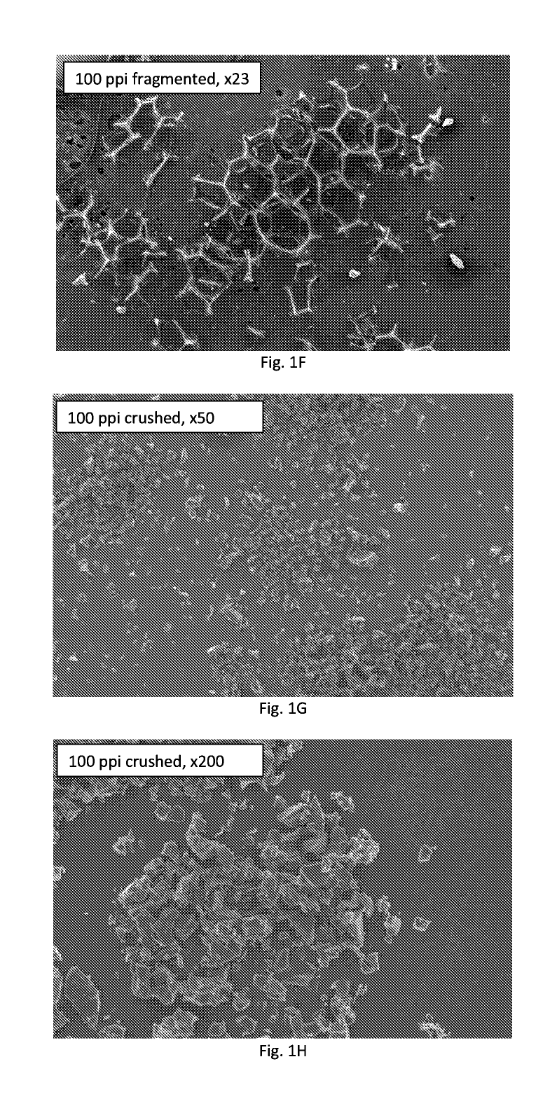

The invention provides carbonaceous particulates optionally dispersed in a carrier matrix for bone and/or soft tissue regeneration. As used herein, “carbonaceous particulates” refers to 3-dimensional particulates consisting of carbon atoms, and specifically carbon allotropes such as amorphous carbons, vitreous carbons, pyrolytic carbons, graphitic carbons, and combinations thereof, but expressly excludes carbon fiber and carbon nanotubes. In some embodiments, the particulates are produced by crushing or fragmenting a porous carbonaceous precursor material into discrete, free-flowing particulates or granular pieces. In some embodiments, the particulates can be derived from fracturing carbon foam structures. For ease of reference, unless otherwise specified, the term “carbon foam” is used herein, in preferred embodiments, to describe vitreous carbon foams but also to include graphitic and pyrolytic carbon. All carbon foams share a common morphology that includes three-dimensional bonding between carbon atoms to form nodes and ligaments, which are three dimensionally connected, creating an open cellular structure. The cell walls, nodes, and ligaments together create the interconnected network (i.e., continuous phase) of the foam matrix, which defines the open spaces of the matrix, with the pores being those open spaces or holes formed through the cell walls. See FIG. 1A for visual reference. Suitable carbon foams for use in the invention include, without limitation, pyrolytic carbon foams, vitreous carbon foams, activated carbons and the like. The carbonaceous matrix has an open cell structure, and more preferably is a reticulated vitreous carbon foam. Reticulated vitreous carbon foams have interconnected pores throughout the matrix which allow the passage of gas or fluid through the open spaces from one cell to the next, as opposed to closed cell structures in which the cells are totally enclosed by their walls and do not have interconnected openings. It will be appreciated that a combination of open and closed cell structures could also be present in the precursor carbonaceous material.

Regardless, the porous carbonaceous precursor material is pulverized, crushed, or otherwise fragmented into discrete particulates, such that the individual particulates in the invention are no longer themselves characterized as have a porous matrix structure. In other words, the resulting particulates are discrete, solid (monolithic) bodies without macro-porosity in contrast to the original, continuous, porous material. When combined, mixed, and/or aggregated together, the intentional aggregation of particulates does, however, re-create interstitial spaces among and between the individual, discrete particulates which generates an artificial “porosity” in the aggregated mixture. This artificial “porosity” contributes to substantial gas/liquid flow and cellular penetration when confined (as in a bony void or defect). The resulting particulates preferably consist essentially and even consist of the carbonaceous material.

The particle size of the resulting particulates will depend to a certain extent on the pore size of the precursor carbonaceous material, as well as the extent to which the precursor material is subjected to pulverization. That is, the particulates can be formed simply by breaking down, fragmenting, or fracturing the precursor material along the ligaments of the matrix destroying the porous structure yielding irregular, non-porous carbon foam fragments (with struts/ligaments extending from the particular body, but like the shape of a toy jack). However, the fractured non-porous carbon foam pieces could be further pulverized into smaller and smaller individual pieces, and eventually into a powder (which may be of a more uniform, regular particle shape and size). Thus, the term “particulate,” as used herein encompasses larger, more granular-sized non-porous bodies, as well as smaller powder-like non-porous particles of carbonaceous material. Likewise, it should be noted that the terms “particulate” and “granule” may be used interchangeably herein and do not necessarily refer to particulates of a specific size, unless otherwise noted. See FIG. 1B.

In one or more embodiments, the process includes first crushing or fragmenting the porous carbonaceous precursor material into discrete, free-flowing particulates or granular pieces of the desired size. In one or more embodiments, the precursor carbonaceous material can be crushed and fragmented using a transiently applied, vertically-oriented force applied to the carbonaceous material between two opposing surfaces. In one or more embodiments, particulates are derived using low-pressure fragmentation by applying pressure to a monolithic carbonaceous porous body between two or more opposing surfaces. This approach typically yields irregular fragments having approximate dimensions of 50-1,000 μm depending upon the porosity (and thus strut/ligament dimensions) of the precursor carbon foam material selected (see Table I).

TABLE I
Nominal dimensions of particulates derived from compression
fracture of 45, 80 and 100 ppi vitreous carbon foam
(DuoCel, ERG Aerospace, Oakland, CA).

| Carbon Foam Porosity (pores per inch) | 45 ppi | 80 ppi | 100 ppi |
|---|---|---|---|
| Maximum Mean Diameter (range) | 560 | 321 | 139 |
| Standard error of the mean | 202 | 168 | 64 |
| Minimum Mean Diameter (range) | 153 | 93 | 50 |
| Standard error of the mean | 69 | 35 | 12 |
| Area (um²) | 0.085 | 0.031 | 0.0072 |

Units of length are given in microns (μm). Statistically significant differences between all measurement groups were at P < 0.0001 (paired t-test). The “diameter” refers to the maximum surface-to-surface dimension of the particulate (i.e., the largest dimension of the particulate).

Thus, particulate sizes for irregular, non-porous carbon foam fragments can be up to about 1000 microns, all the way down to about 40 microns. It will be appreciated that various particulate dimensions can be achieved by the selection of specific carbon foam precursor morphologies having appropriate dimensions (porosity, ligament length) to yield fractured pieces of the target size (which may or may not be followed by further pulverization of the initially fractured foam).

In other embodiments the precursor carbonaceous material can be subjected to shearing forces such as can be produced by placing the material between two opposing surfaces that are moved in opposite directions horizontally as force is applied to the carbon. This procedure results in particles having a broader size range of larger-dimensioned particulates having greater irregularity in shape. In other embodiments, the resulting particulates can be further subjected to pulverization, using a mortar and pestle or similar mechanism to yield a fine carbon powder of smaller, uniform dimensions. Regardless of the embodiment, this pulverization process can be followed by sorting and grouping the materials by size, such as sieving in order to derive discrete size ranges that are appropriate for the desired application.

Exemplary carbon foams for use as precursors in the invention are commercially available from various manufacturers, including, without limitation, ERG Aerospace Corp., Oakland, Calif. (DUOCEL® RVC 45, 60, 80, and 100 vitreous carbon foam); GrafTech International, Parma, Ohio (GRAFOAM® FPA-02, -05, -10, -20, and -35 graphitic foam); PocoGraphite, Inc., Decatur, Tex. (POCOfoam® graphitic foam); Koppers, Pittsburgh, Pa. (KFOAM® L1, L1a, and D1 graphitic foam); Touchstone Research Laboratory, Ltd., Triadelphia, W. Va. (CFOAM® 20, or 25); and Ultramet, Pacoima Calif. (RVC 65 PPI, 80 PPI, RVC with CVD materials, and RVC with integrally bonded coating). Methods for making carbon foams are also known in the art, including U.S. Pat. Nos. 6,103,149, 6,656,238, 6,656,239, and 6,749,652, incorporated by reference herein to the extent not inconsistent with the present disclosure.

Additionally, it can be understood that other non-foam, bulk carbon materials could be used as precursors for fragmentation if other shapes or sizes of carbon particles are desired. As the particle size is decreased, the particle shape is less and less dependent on the shape of the bulk precursor, and the molecular structure of the material itself becomes more important. For use in the invention a plurality of fairly uniform, monodisperse collections of particulates can be used in combination with osteogenic components to promote bone growth. Alternatively, a plurality of particulates of varying particle size can be combined together (polydisperse collection) with osteogenic components to form a morphologically-heterogeneous graft. It is also envisioned that carbonaceous particulates for use in the invention include materials that have been synthesized or otherwise originate in particulate form (rather than being created through grinding or pulverization of a larger precursor structure). Such materials could be created through extrusion, 3-dimensional printing, and the like.

In one or more embodiments, the carbonaceous particulates are dispersed in a carrier matrix that enhances cohesion and malleability to yield a flowable composite composition. Exemplary carriers include any biocompatible and biodegradable matrix material, such as a gel or hydrogel-type matrix (or gel or hydrogel precursor that is subsequently gelled), in which the particulates can be dispersed to yield a self-sustaining body. The term “self-sustaining body” means that the composite retains its shape without an external support structure, and is not susceptible to deformation merely due to its own internal forces. However, the self-sustaining body is “flowable,” which means that it is pliable, and deformable, like a gel, putty, or paste, without fracturing or cracking. In other words, the self-sustaining body will not recoil or spring back into shape after bending, stretching, flexing, and/or compression. Thus, when an external pressure or force is applied to the self-sustaining body, it can be shaped, molded, formed, extruded, and/or injected, as appropriate, for the target site of implantation. In one or more embodiments, the flowable composite composition is syringeable, meaning that it can be drawn into and/or dispensed from a syringe. Suitable carriers can comprise, without limitation, gelatin, carboxymethyl cellulose, hyaluronic acid, phosphatidyl choline, atelocollagen (solubilized collagen), and the like. The weight ratio of carbon particulate to carrier can vary widely, with exemplary ranges including from about 1:2 to about 1:10, and preferably about 1:4.

In one or more embodiments, the carrier matrix can also comprise optional cohesion agents. Examples of natural cohesion agents include, without limitation, collagen (e.g., shredded), albumin, non-crosslinked collagen fibers, chitin, chitosan, silk, carbon fibers, and the like. Demineralized bone matrix (DBM) can also be used as a cohesion agent (as well as an osteopromotive agent). When present, the weight ratio of carrier to cohesion agent will be from about 0.5:1 to about 1:5, and preferably about 1:1.

Synthetic colloids, hydrogels or cryogels comprised of polymers such as polyvinyl alcohol, polyurethane or polyacrylamide can also be produced with carbon particulates with or without pre-adsorption with a therapeutic component. It can be readily appreciated that carbon particulates embedded in polymeric materials can be delivered in surgical procedures without either a carrier or cohesive agent. These wholly synthetic implant materials could, by varying reaction conditions, take the form of rigid sheets or slightly viscous but coherent gels that can be delivered via ejection from a syringe.

In one or more embodiments, the composite can further comprise an aqueous salt solution such as phosphate buffered saline, physiological saline, Hank's balanced salts, or deionized water, or other biocompatible solvent system to assist with dispersing and wetting the composite mixture (i.e., the carbonaceous particulates, carrier and optional cohesive agent) uniformly throughout the matrix. When present, the weight ratio of carrier to buffer solution will be from about 0.5:1 to about 5:1, and preferably about 2:1.

In an alternative embodiment, instead of being dispersed in a carrier matrix, the carbonaceous particulates can be enclosed within a biocompatible mesh container. The mesh size of the container will be selected depending upon the smallest particle size of the particulates. Examples of suitable biocompatible mesh materials include polyethylene terephthalate (PET) mesh, polyester mesh, and the like.

Regardless of the embodiment, the carbon particulates are combined with one or more osteopromotive and/or therapeutic agents. In one or more embodiments, carbon particulates are mixed with therapeutic and/or osteopromotive agents (which may themselves be in particulate form). In one or more embodiments, a coating, film, or monomolecular layer of a therapeutic and/or osteopromotive agent can be immobilized on the surface of the individual carbon particulates. “Osteopromotive” agents are materials that facilitate or enhance bone formation. It will be appreciated that a given material can be both osteopromotive and therapeutic, and these categories are descriptive and not necessarily mutually exclusive. Osteopromotive materials include both osteoinductive compositions and osteoconductive compositions. By comparison, osteoconductive compositions are ones that permit and even enhance bone growth over the surface of the material (yet often require an additional osteoinductive signal), and include allogenic or autogenic bone fragments, calcium phosphate, bioactive ceramics (e.g., synthetic hydroxyapatite, coralline), sintered bone (Bio Oss®), porous polycaprolactone, and combinations thereof. Osteoinductive materials stimulate osteoprogenitor cells to differentiate into osteoblasts that then begin new bone formation, and include cytokines, demineralized bone matrix (DBM), and the like. Therapeutically-useful cytokines include bone morphogenetic proteins (“BMPs”), insulin-like growth factors, vascular endothelial growth factors, parathyroid hormone, platelet-derived growth factors, epidermal growth factor, fibroblastic growth factors, and the like. Useful biologic therapeutics that can be used with the carbon particulates include heparin, chondroitin sulfate, platelet-rich plasma, bone marrow aspirate, or mesenchymal stem cells. It can be appreciated that some of these therapeutics can be premixed with the carbon particulates and/or carrier, and stored for later use while other, more labile or autologous materials, can be introduced at the point of care. Other osteopromotive agents suitable for use in the invention include nucleic acid vectors, (e.g., plasmid DNA, cDNA, microRNA, lgRNA, siRNA, adenovirus-associated or RNAi constructs) harboring nucleic acid sequences, encoding proteins, or RNAs known to be osteopromotive from in vitro or preclinical studies. Other therapeutic agents for use in the invention include small molecule drugs as well as biologics. Exemplary small molecule drugs include antibiotics (e.g., vancomycin, tobramycin, gentamicin, nanoparticulate silver), anti-inflammatories (e.g., COX-1, COX-2, steroidals), and anti-coagulants (e.g., conjugated heparins, warfarin). An additional class of therapeutics includes small molecules known to be synergistic or agonistic with regard to therapeutic signaling pathways. Exemplary therapeutic biologics include proteins isolated from animal tissues such as collagen, fibrin, fibrinogen, vitronectin, and immunoglobulins, as well as synthetic, therapeutic monoclonal antibodies.

It will be appreciated that many of the above materials may be used alone or in combination. It will also be appreciated that the above materials can be combined with osteogenic bone-forming strategies involving the ex vivo manipulation, material adhesion, replication and differentiated function of exogenous cells to the osteoconductive materials discussed above. For example, explanted, minimally-manipulated cells from either the patient (autograft) or donors (allograft) can be used to enhance bone growth, including adult stem cells (e.g., bone marrow, adipose, umbilical cord blood, etc.), bone-derived osteoblasts, chondrocytes isolated from cartilage, and complex, undefined cell populations current in clinical use, including bone marrow aspirate and platelet-rich plasma. Genetically manipulated allogeneic, immunocompatible cells expressing osteoinductive growth factors, cytokines or cell attachment proteins may also have therapeutic potential in the context of the materials described herein.

In use, the particulates are first soaked in 100% ethanol to remove any residual surface contaminants left behind by the manufacturing and handling procedures and then sterilized either by exposure to 70% ethanol or steam autoclaved. Following surface preparation, the carbonaceous particulates are ready to be combined with the osteopromotive and/or therapeutic agents. In some embodiments, the particulates are prepped for adsorption of osteopromotive and/or therapeutic agents that can be efficaciously applied to a wound or defect site. The most direct method for applying the materials to the particulates is by direct contact with the carbon particulates in an aqueous solution that has been optimized with respect to therapeutic concentration, pH, ionic strength, and inclusion of others solutes such as salts, surfactants, complex carbohydrates, glycosaminoglycans, lipids, and the like.

For loading of therapeutic and/or osteopromotive agents, such as rhBMP-2, for example, the pre-sterilized particulates are first rinsed in the same solvent system to be used to deliver rhBMP-2. Next, the rinsed particulates are contacted with a solution containing the specified concentration of therapeutic and/or osteopromotive agent. The adsorption of some therapeutic and/or osteopromotive agents, such as rhBMP-2, is very rapid (requiring at least 30 minutes of exposure, or preferably 12 hours exposure) while other therapeutic and/or osteopromotive agents such as non-TGF-beta cytokine proteins and small molecule therapeutics my require subsequent lyophilization of the therapeutic and/or osteopromotive agent solution onto the surface of the particulates. The resulting treated particulates are ready for use without further processing, for example as implants for animal studies or with approved implants in orthopedic or wound therapy procedures where the particulates are constrained within a mesh pouch or other suitable containment.

In alternative embodiments, uncoated or coated particulates can also be dispersed in a carrier to create a composite as described herein. Advantageously, the composite can be created, for example by the surgeon, immediately prior to the time of use (e.g., during surgery). Alternatively, the composite can be lyophilized and then rehydrated at the time of use, or can be stored frozen (preferably at −80° C.) until use, depending upon the physical and chemical characteristics of the therapeutic and/or osteopromotive agent.

In one or more embodiments, the invention leverages the unexpected finding that BMPs and other biologically-active osteopromotive materials and/or therapeutic agents, such as therapeutic cytokines and passivating blood proteins, adhere to these carbonaceous matrices with a tenacity able to withstand rinsing (irrigation) and further that the bound substances with defined functionality retain their biological activity (as tested by challenging responding cell lines). This means that when the composite is implanted, the coating is resistant to being immediately released from the particulates and migrating away from the site of implantation. These properties give rise to a carbon-based implant that delivers a small burst of active agent sufficient to promote stem cell recruitment to the implant site while retaining the bulk of the active agent coating in situ over a sufficient period of time to provoke cellular differentiation down pathways that are osteopromotive. In particular, the data demonstrates that adsorptive binding of an osteopromotive cytokine (e.g., rhBMP-2) to the carbon foam scaffold exceeds absorptive binding (which results from simple infiltration of therapeutic solution into the carbon foam). The importance of this unexpected outcome is that it yields an additive effect on new bone formation that is greater than the concentration of the osteopromotive agent alone due to the two distinct pharmacokinetic release profiles. The first of these is a relatively rapid burst of release of the absorbed osteopromotive fraction followed by a second, much slower release profile from the adsorbed fraction bound to the surface of the particulate.

In one or more embodiments, the coating is immobilized on the surface of the carbon foam particulates, but preferably is not covalently or otherwise chemically bonded thereto. Thus, the coating material is physically immobilized, relying instead on van der Waals or ionic-type attraction between the carbon foam particulate surface and the coating material. This provides a significant advantage in that the osteopromotive or therapeutic agents do not have to be chemically modified (for covalent bonding), and thus retain their full bioavailability when implanted into a patient, even though tightly bound to the particulates. Advantageously, the use of the coating in combination with the carbon foam particulates permits not just in-growth of bone, but eventual through-growth of bone throughout the entirety of the composite.

The carbon foam particulates are characterized by the ability to retain proteins adsorbed onto the surface of the particulates. More specifically, when subjected to a protein elution test, the particulates have the ability to retain at least about 50% of protein, more preferably at least about 7% of protein, and even more preferably at least about 900% of protein initially contacting the particulate. That is, when the carbon particulate is placed in contact with a solution containing a protein, such as BMP, at least about 50% of the protein from the free solution will be taken up and retained by the foam and resulting particulates (preferably at least about 70% and more preferably at least about 90%), based upon the total amount of protein in the solution taken as 100%. In other words, the protein content of the solution is decreased by at least about 50, 70, or preferably 90% after immersing the particulate into and then removing it from the solution. Protein retention can be tested using a protein elution test as described in US 2012/0185047 and US 2012/0095558, incorporated by reference herein.

The protein elution tests are multi-partite and address important functional issues of productive contact between osteoinductive material and conductive carbon foam particulates, the degree of persistence of the binding interaction, the percentage of coating released to the surrounding environment, and percentage retained in a biologically-active, bio-available form. An example of one such comprehensive analysis involves contacting selected foam materials with a cytokine solution of known concentration either by passage of the solution over the solid object or by low pressure vacuum loading. The concentration of the remaining wash solution following completion of a specified loading time is reevaluated by enzyme-linked immunosorbent assay (ELISA) to determine the cytokine depletion and thus de facto transfer of cytokine to the carbon foam. Replicate loading studies using recombinant human BMP-2 (rhBMP) reliably yielded depletion values of at least 50%, that is, simple contact between solute and carbon material yields transfer of 50% of the cytokine to the carbon foam from solution. Verification of this presumptive loading percentage can be validated using one of two methods: 1) sandwich ELISA of protein stripped from coated carbon foams with chaotropic agents (e.g., 4M guanidine hydrochloride) or reagents that interfere with ionic interaction (e.g., 5M NaCl; 0.5M glycine, NaOH, pH 10); or 2) Solid-phase ELISA of cytokine adsorbed to carbon matrix using BMP-2 antibody conjugated to biotin or chromogenic-generating enzyme providing quantification of the amount of BMP-2 adsorbed onto the material surface.

Bound BMPs (such as BMP-2 or other cytokines described herein) can also be evaluated by the criterion of biological activity by exposure of cells lines that are competent to respond to the adsorbed cytokine in question. That is, cells exposed to cytokine-coated particulates can readily be induced to develop novel phenotypes such as a bone-forming osteoblast that can be measured using a combination of enzymatic, molecular (real time PCR), and histochemical assays. This is important for retention of biological activity (rather than migration away from) at the clinically-relevant site of implantation.

In addition, the particulates have a remarkable ability to retain the coating after implantation, drawing the components of the bone regeneration process into the implant. This is particularly advantageous for the use of proteins such as BMP, which are retained by the particulates instead of being released into surrounding tissue, and avoids the formation of ectopic bone or unwanted bone overgrowth. After implantation, the particulates will preferably retain at least about 75%, more preferably at least about 80%, even more preferably at least about 90%, and most preferably at least about 95% of the immobilized protein, based upon the total initial protein content of the coating taken as 100%. This can be tested in vitro for a given matrix prior to implantation using a protein release test. Thus, when subjected to a protein release test for a period of about a week, implants according to the invention will have high retention and low release of bound protein. Thus, the rate of mineralization can be reliably controlled using the inventive composite, due to the linear relationship between the amount of osteopromotive material (such as BMP) present in the implant, and the mineralization rate. Accordingly, a given amount of protein can be loaded onto the particulates before implantation to achieve a target level of mineralization, since very little protein is lost to surrounding tissue.

This is an important and unexpected development. Its importance lies in the necessity for such osteopromotive agents to be released in a rapid burst followed by a slower sustained release in order to achieve optimal clinical efficacy as is generally acknowledged in the literature. For example, with respect to cytokines, the initial release of cytokine from an implant site (into which the composite has been implanted) creates a concentration gradient of cytokine signal that serves to guide stem cells and differentiated osteoblast to the wound site. The subsequent slow release component serves to initiate multiple signaling pathways that lead to osteoblast differentiation of stem cells now residing in contact with the composite surface.

As mentioned, however, additional embodiments of the invention are directed towards mixing of the carbon particulates with a suitable osteopromotive and/or therapeutic material, which may itself be a particulate, but can also be in a liquid, gel, or other form, or dispersed in the same. Such embodiments do not necessarily require coating and/or adsorption of the osteopromotive and/or therapeutic material on the particulate surface (although such may occur nonetheless). The carbon particulates and osteopromotive and/or therapeutic agents can be dispersed in an appropriate carrier matrix to create a composite. The carbon particulates and osteopromotive and/or therapeutic agents can be pre-mixed before being dispersed in the carrier, or they can be sequentially or simultaneously (but individually) dispersed in the carrier to create the composite.

Methods of using the inventive carbon foam particulate-based implants are also described herein. The methods generally comprise providing a carbon foam particulate mixture dispersed in a carrier matrix or enclosed within a biocompatible mesh retainer. For example, the implant is provided to a surgeon or other medical or clinical professional for placement, insertion, or fixation in a bone void or site of tissue damage. The inventive implant can be used as a bone void filler for virtually any type of voids, defects, or gaps in bone, whether due to surgical resection, trauma, disease, pathologic anatomy, etc. More specifically, the implant can be used as a bone graft scaffold for replacement and/or repair of portions of bones, calvarial defects, osteochondral defects, fractured vertebrae, intervertebral or posterolateral spinal fusion, alveolar ridge augmentation, extraction socket restoration and the like, and or as a scaffold for repair of soft tissues. Thus, it will be appreciated that the implant can be shaped to fit cavities or depressions in the bone, holes present through a bone, damaged or missing end sections, and the like. The implant can be formed, shaped, or injected/syringed by the surgeon to fit the exact shape and size of irregular voids and provide a precise interface with undamaged bone adjacent to the void (similar to conventional bone cement). Depending upon the carrier matrix used with the implant, the implant can then be allowed to “set” and harden. Alternatively, the implant may remain somewhat pliable through the regeneration process.

In one or more embodiments, the composite implant is provided to the surgeon, preferably with instructions regarding shaping the implant to fit the void and fixation of the implant in the void. Such a kit could also optionally include tools for injecting, or extruding the implant, as well as appropriate tools and fasteners for fixing the filler in the void. After the implant is shaped and sized to the bone void, it can be placed in the void and fixed using suitable techniques including pins, screws, friction-fit engagement, sutures, adhesives, suturing the patient's own skin, suturing to other surrounding soft tissues such as fascia or connective tissue, and/or combinations thereof. In one or more embodiments, the implant could also be placed in the void and (further) shaped or contoured, if necessary, before and/or after fixation. Likewise, it will be appreciated that the implant material itself can be used in percutaneous applications via syringe needle injection for simple orthopedic procedures not requiring open surgery.

Further, carbonaceous particulates embedded in a suitable therapeutic scaffold such as hemostatic sponges would be useful for wound care, or procedures requiring slow delivery of a protein or small molecule therapeutics. Further uses include posterolateral spinal fusion, where the carbon foam particulate mixture dispersed in a carrier matrix or enclosed within a biocompatible mesh container is laid over the transverse processes of vertebrae to be fused. The composite could also further be combined with other scaffolds known in the art, such as collagen sponges, fibrin gel, and synthetic polymers including polycaprolactone, polylactide, poly(lactic-co-glycolic acid), and polyglycolic acid, wound care pads, and the like.

This adaptable/adaptive therapeutic modality can potentially be customized to address any of a number of current needs in regenerative medicine including:
Spinal fusion (carbon particulate mixture encased in PET mesh or equivalent; composited with flowable carrier formulations to confer syringe application of materials to intervertebral ring devices);
Long bone defects (carbon particulate mixture in flowable form deliverable by syringe in open surgeries or percutaneously with syringe-fitted needle);
Filling either bony defect voids or tooth extraction sockets for implant placement (either PET mesh-constrained or flowable carbon particulate mixture formulations);
Application of carbon particulate mixture in either flowable formulations (syringe delivery) or embedded in manufactured collagen or synthetic polymer sheets (such as polyvinyl alcohol, polyurethane, or cross-linked styrene) for direct application (diabetic foot ulcers or other chronic wounds) or surgical implantation in contact with vascular, cardio-myocyte, liver, kidney or other tissue characterized by extracellular matrix;
Restoration of bony anatomy where a mixture of carbon particulates and biologically-active demineralized bone matrix are used to provoke bone formation;
Bone formation at “heterotopic” sites (such as in posterolateral spinal fusion), using carbon particulate/demineralized bone matrix composites.

The invention also contemplates use of the carbon particulates in alternative carrier matrices, such as in curable polymer matrices. Such curable polymer matrices include any biologically-compatible polymers that can be cured through exposure to activating radiation (e.g., UV, infrared), or through thermal heating to create a somewhat rigid cured structure. For example, the carbon particulates could be used as part of a curable matrix to create a custom patient-specific 3-dimensional shape for bone repair. Likewise, the carbon particulates can be embedded into polymer sheets that can be used for bone repair.

Regardless, once the carbon particulate material is implanted, osteogenesis begins, which involves bone and tissue ingrowth into the carbon particulate, including the establishment and maintenance of a vascular bed in the carbon particulate implant, eventually resulting in new bone formation (mineralization). Unlike many existing scaffolds, the patient's vascular supply is able to readily penetrate through the carbon particulates of the implant, which supports the development of new blood vessels that are essential to bone repair and allows the formation of a robust vascular bed. Advantageously, the inventive implant supports vascularization throughout the entirety of the carbon particulate mixture, resulting in not just in-growth of bone tissue at the exterior surface of the implant (1-2 mm), but “through-growth” of bone tissue through the entire implant body. As bone tissue infiltrates the matrix and mineralizes, the carbon particulate mixture and implant is slowly degraded and absorbed by the patient's body. More preferably, at about 6 weeks after implantation, the carbon particulate mixture is preferably at least about 75% resorbed, more preferably at least about 85% resorbed, and even more preferably at least about 95% resorbed. In other words, at about 6 weeks after implantation, the treated area will comprise less than about 25% carbonaceous material from the implant, more preferably less than about 15% carbonaceous material, and even more preferably less than about 5% carbonaceous material, based upon the total initial carbon content of the implant taken as 100%. The terms “resorption” and “bioresorption” are used interchangeably herein and mean that the material is broken down by the body over time through cellular activity or chemical degradation and does not require mechanical removal from the body. A particularly unexpected aspect of the invention is that direct displacement/replacement of the implant by the new tissue will preferably be observed, as opposed to overgrowth of tissue. This through-growth of bone in alignment with the original implant position is a surprising and particularly advantageous aspect of the present invention.

It will be appreciated that the inventive implant will have additional uses beyond traditional bone void filling, including arthroplasty, fracture repair, and reconstructive surgery. The implant could also have applications in plastic and cosmetic surgery where bone modifications (that include restoration or augmentation) are required. As noted above, the implant can also serve as a drug delivery device for orthopaedic applications, providing local delivery of therapeutic agents, osteogenic factors (such as BMPs and other biological response modifiers), and factors to inhibit bone resorption (such as bisphosphonates).

It will be appreciated that not only bone grafting procedures but also other soft tissue regenerative procedures requiring grafting are substantially enhanced and addressed through this invention. Specifically the enclosure of a carbon foam particulate mixture within biocompatible meshes, incorporation of granules into collagen fiber matrices, as well as flowable embodiments created by compositing with agents that confer viscosity or miscibility to the solid particulates confer adaptability of the therapeutic component to a multiplicity of voids, lesions, defects, and other anatomic areas requiring defect-conforming therapeutic grafts.

Additional advantages of the invention will be apparent to those in the art upon review of the disclosure herein and the working examples below. For example, the invention described herein is discussed primarily with respect human-based therapies; however, it will be appreciated that the treatment can be applied for clinical research or therapeutic treatment to any suitable animal, including, without limitation, dogs, cats, and other pets, as well as, rodents, primates, horses, cattle, etc.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Osteoinductive Properties of Vitreous Carbon Foam Materials

### 1. Materials and Methods

In this Example, 6×5 mm carbon foam discs (DUOCEL®, ERG Aerospace) prepared from bulk materials were sterilized in 70% ethanol and exhaustively rinsed in phosphate buffered saline (PBS). For BMP-2 loading studies, foam disc were submerged in PBS containing specified amounts of cytokine for 24 hours. Unbound cytokine was separated from the foam-bound fraction by low-speed centrifugation, followed by 3 rinses in PBS. The BMP-2 that was not adsorbed by the carbon foam was measured in the residual soak and subsequent wash solutions using a sandwich ELISA assay.

The rinsed discs containing adsorbed BMP-2 were then placed into wells of a tissue culture plate and C2C12 mouse myoblast cells were added to the carbon matrix by direct pipetting. Carbon materials pre-incubated in PBS containing 50, 100 or 200 ng/ml BMP-2 comprised the experimental study groups. Cell culture medium was added to the plates containing the cells and carbon matrix following a 2 hr attachment. Negative control C2C12 cell cultures received no additional material while positive controls received soluble BMP-2 applied to cells already adhered to tissue culture polystyrene. Four days later, rinsed cell monolayers or three dimensional cultures on carbon foam were lysed and alkaline phosphatase activities determined in the cell extracts. Alkaline phosphatase specific activities were determined by normalization against cell numbers in parallel cultures using an MTT cell viability assay.

The osteoinductivity of DUOCEL® carbon foam was evaluated by material infiltration of C2C12 mouse myoblasts or NHOSC human osteoblasts into rinsed carbon foam discs followed by measurement of alkaline phosphatase induction. Control cell cultures were 2-dimensional cell monolayers on tissue culture polystyrene. Enzyme specific activities were determined at 2, 4, and 6 days (C2C12) or at days 3, 5 and 7 days (NHOSC cells).

### 2. Results

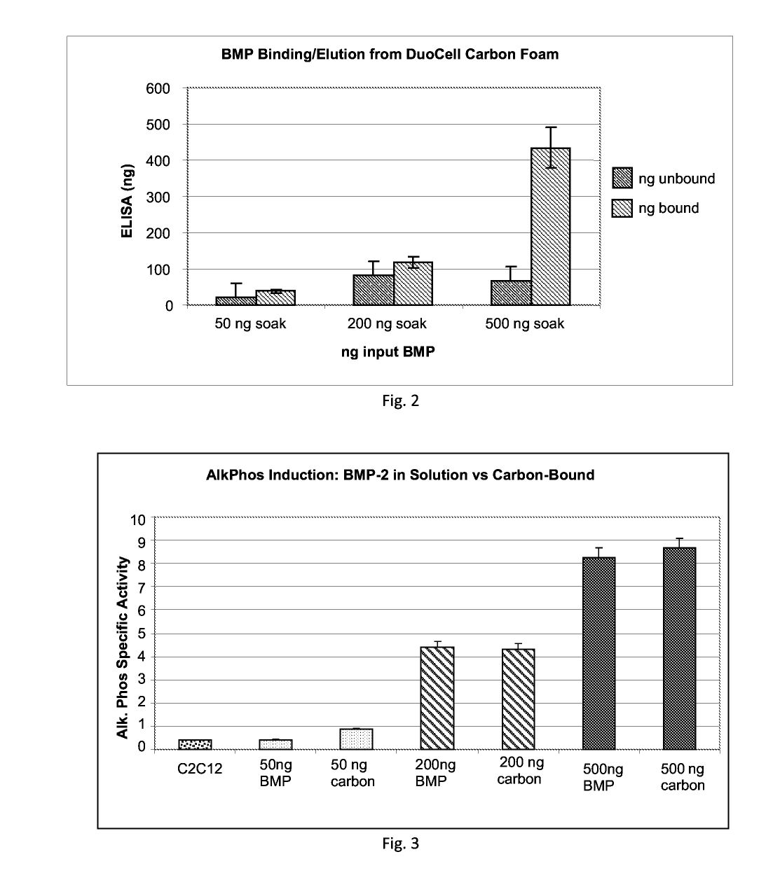

A typical outcome of a BMP-2 adsorption assay using DUOCEL® carbon foam is depicted in FIG. 2. Unbound values were derived by ELISA and bound values by subtraction of unbound from input. At lower BMP-2 inputs (50 and 100 ng/ml) the adsorbed and non-adsorbed fractions were approximately equal. At a higher BMP-2 input (500 ng/ml) the adsorbed:nonadsorbed ratio tended to increase from 1:1 to a value of circa 5:1. Aggregate results of several trials show BMP-2 concentration-dependence of binding at constant carbon foam mass input (data not shown).

To assess both the relative amount of BMP-2 bound to the carbon discs and whether biological activity is retained following adsorption, C2C12 myoblasts were exposed to rinsed discs and alkaline phosphatase activity was measure 4 days later (FIG. 3). This mouse myoblast cell line is capable of acquiring a bone cell (osteoblast) phenotype upon exposure to osteoinductive cytokines (including BMP-2) and thus provides a valid bioassay system for cytokine-provoked cell signaling. Alkaline phosphatase induction provides a reliable phenotypic marker of a commitment to the osteoblast phenotype in cells so provoked. Significantly, carbon foam discs pre-adsorbed in the indicated BMP-2 solutions induced an amount of alkaline phosphatase activity that was identical to positive control C2C12 cell culture receiving the same amount of soluble cytokine. This outcome indicates that the levels of alkaline phosphatase activity induced by treated carbon foam is the resultant of both adsorbed cytokine and intrinsic material osteoinductivity since the amount of BMP-2 retained by the discs was only approximately 50-80% of the original soak solution (FIG. 2).

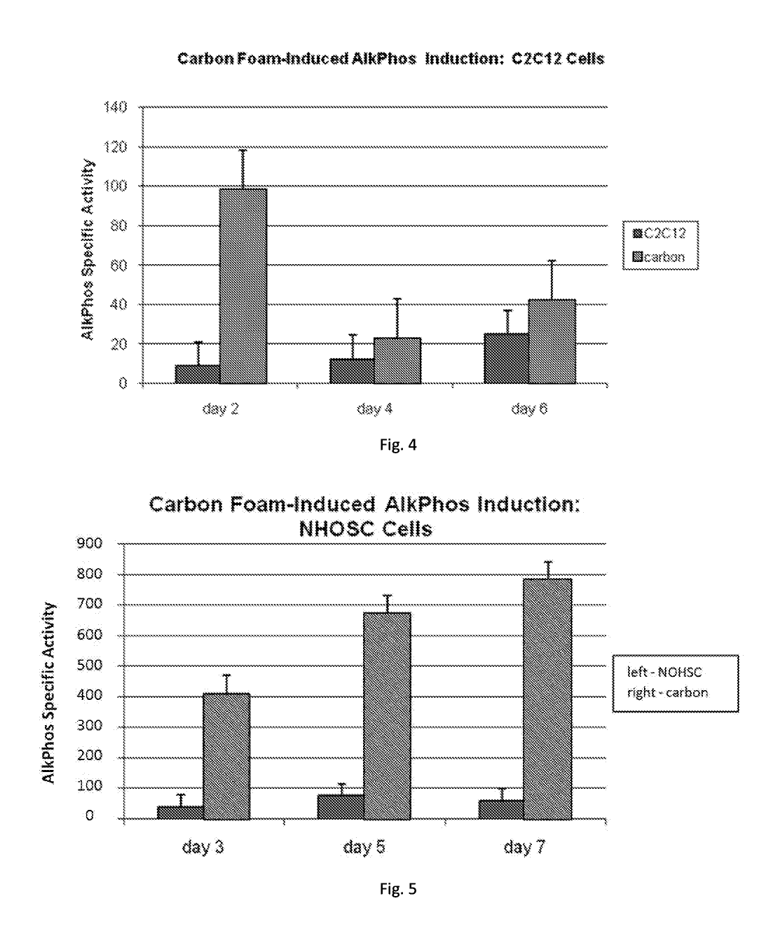

Alkaline phosphatase induction experiments (over a 6-7 day period) were also conducted using DUOCEL® carbon foam following exposure of mouse C2C12 myoblasts and human NHOSC osteoblasts (FIGS. 4 and 5). The former cell line addresses the ability of a predetermined cell type to re-differentiate and the latter the ability of an osteoblast to up-regulate a specific cellular phenotype. Both cell lines challenged with biomaterial exhibited increases in alkaline phosphates specific activity. The C2C12 cells trended towards higher activities than controls at all time points but was statistically significant from controls at day 2 only (FIG. 4). In contrast, the NHOSC cell line responded to carbon foam by synthesizing 8- to 10-fold higher levels of alkaline phosphatase that was statistically significant across the entire time interval (FIG. 5). Parallel analyses of enhanced osteoblast-like gene expression by RT-PCR have revealed similar trends (data not shown).

### 3. Conclusions

Carbon foam materials incorporate physical properties that mimic natural bone. Chief among these include mechanical rigidity, pore diameter, and pore interconnectivity, properties that contributed to the significant bone forming tendencies documented in the present studies. These tendencies include both the propensity of the materials to adsorb BMP-2 in a signaling-competent chemical form and the provision of a three-dimensional framework that allows bone cell (osteoblast) phenotypes to be expressed even in cells displaying a pre-determined phenotype. These two outcomes are consistent with the historical scientific literature pertaining to the topic of bone cell differentiation and concomitant predictors of materials and medical devices whose clinical outcomes culminate in significant bone formation. These results demonstrate the osteoinductive biomaterial property of carbon foam.

### Example 2

### BMP-2 Adsorption, Release and Osteoinduction

In this Example, the ability of carbon foam materials to deliver BMP-2 to mesenchymal stem cells in a form promotive of osteoblast differentiation was studied. These initial tests examine two interrelated aspects: quantitative aspects of BMP-2 binding to two carbon foam materials using an ELISA assay; and verification of cytokine binding through ELISA analysis of the amount of presumptive bound cytokine desorbed using chemical means. A preliminary account of stem cell replication and osteoinduction is also presented. DUOCEL® 80 PPI vitreous carbon foam and Kopper KFOAM® 80 PPI graphitic carbon foam was used. The specimens were cut from manufacturer's blocks using biopsy punches into cylinders, 8-10 mm in diameter and 2-3 mm in thickness, depending on test protocol (see below). Additional processing is described below.

### 1. BMP-2 Binding Assay

Carbon foam cylinders were sterilized by immersion in 70% ethanol for 4 hours followed by centrifugation to removed wicked liquid, then air dried in a laminar flow hood for 14 hr. BMP-2 solutions (10, 5 and 2 μg/ml) in sterile water were prepared and 1 ml of each solution was applied to carbon foam constructs in a 12-well cell culture plate. Plates were transferred to a vacuum apparatus at 37° C. and the BMP solution was driven into the foam void volume in vacuo for 24 hr. Excess BMP solution was then removed from the cylinders by centrifugation for 5 min. at 200 rpm and the foams were transferred to clean wells and air dried overnight. The BMP wash solutions (original soak plus 200 rpm void volume fraction) were combined and stored at −20° C. for ELISA estimation of unbound BMP-2 (i.e., “left over” fraction not absorbed by the foam). The dried carbon discs were then taken through 6 cycles of rinsing (by immersion) in 1 ml PBS followed by centrifugation, pooling of eluates, and drying (“days 1-6 eluates”). ELISA assays of BMP-2 standards, soak solutions, post-soak solutions and disc rinses were performed using a sandwich assay. Standard curves were generated using the same commercial BMP-2 product used for binding (eBioscience).

### 2. BMP-2 Elution Assay

Carbon foam discs were pre-absorbed in stock BMP-2 solutions (2 μg/ml) using low pressure vacuum as described above. Hydrated discs were then centrifuged at 200 rpm as above and the removed liquid combined with the original soak solution. This step was followed by two 24-hr PBS rinses conducted as described above. The rinsed BMP-infused discs were then subjected to two separate elution regimes consisting of a 4 hr soak in 1M glycine or a 2 hr soak in 4M guanidine hydrochloride. These solutions are routinely used to solubilize adsorbed, immobilized proteins or proteins embedded in an extracellular matrix. The eluate solutions were combined with liquid expressed by 200 rpm centrifugation and neutralized by the addition of 72 μl 1M Tris to effect a shift to pH 7.5. The neutralized eluate solutions along with control BMP-2 solutions containing glycine or guanidine salts at concentrations equivalent to experimental discs were analyzed by ELISA as described above.

### 3. C2C12 Replication and Osteoblast Induction Assays

Both types of carbon foam discs were ethanol sterilized, wet in PBS and then PBS containing 100 ng BMP-2 was applied directly to the materials. The BMP was allowed to adsorb to the materials for 2 hr at 37° C. Ten thousand C2C12 mouse myoblast cells were then added to empty wells (tissue culture polystyrene) or applied (in a volume of 50 μl) to carbon foam discs with and without preadsorbed BMP. Following a 2 hr attachment of cells at 37° C., all wells received 400 μl DMEM tissue culture medium+10% FBS. At days 1, 3, 5 and 7 post-plating, wells containing the complete data set were processed for MTT and alkaline phosphatase activity assays.

### 4. Results

a. BMP-2 Binding Assay

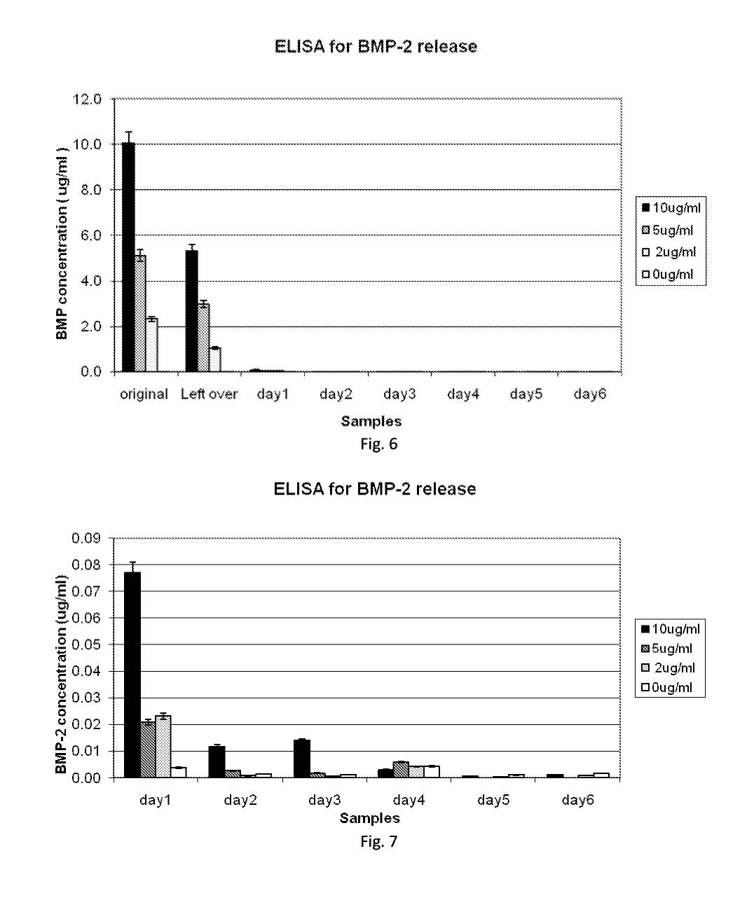

The initial binding assay revealed that approximately one half (˜50%) of the BMP-2 in the soak solution was recovered following the 24 hr exposure to carbon foam (FIG. 6). This result was obtained irrespective to the starting concentration of the BMP-2 stock solution. More precisely, the concentration of the soak solutions was decreased approximately 50% following carbon disc exposure. Significantly, additional PBS rinses designed to elute loosely bound BMP protein or entrapped BM BMP solution resulted in only small incremental detection of released BMP by ELISA over a 6 day period (FIG. 7). The amount of BMP-2 released from each BMP soak concentration amounted only to approximately 1% of the input total.

b. BMP-2 Elution Assay

The results of the BMP-2 elution assays using guanidine-HCl and glycine chaotropes were inconclusive. In both cases preadsorption of BMP-2 at a concentration of 2 μg/ml in the soak solution yielded approximately 50% loading of cytokine onto the material and 50% presumably bound to carbon. BMP-2 was not detected in either desorption solution suggesting that either some proportion of BMP-2 in the soak contacts carbon and is denatured (rendering the cytokine undetectable by BMP IgG in the ELISA) or that BMP is indeed bound but irreversibly so.

c. C2C12 Cell Replication and Alkaline Phosphatase Induction Assays

We have been interested in establishing baseline data for cell replication and osteoblastic phenotype development in response to contact with carbon foam materials. We incorporated BMP in these studies as a positive control for osteoblast marker induction as well as to determine if synergy with carbon materials (positive of negative) was taking place. This preliminary study included one representative each of the vitreous (DUOCEL®) and graphitic (KFOAM®) carbon materials. C2C12 myoblasts were chosen for these preliminary assays owing to their multipotent capabilities including osteoblast phenotypic acquisition.

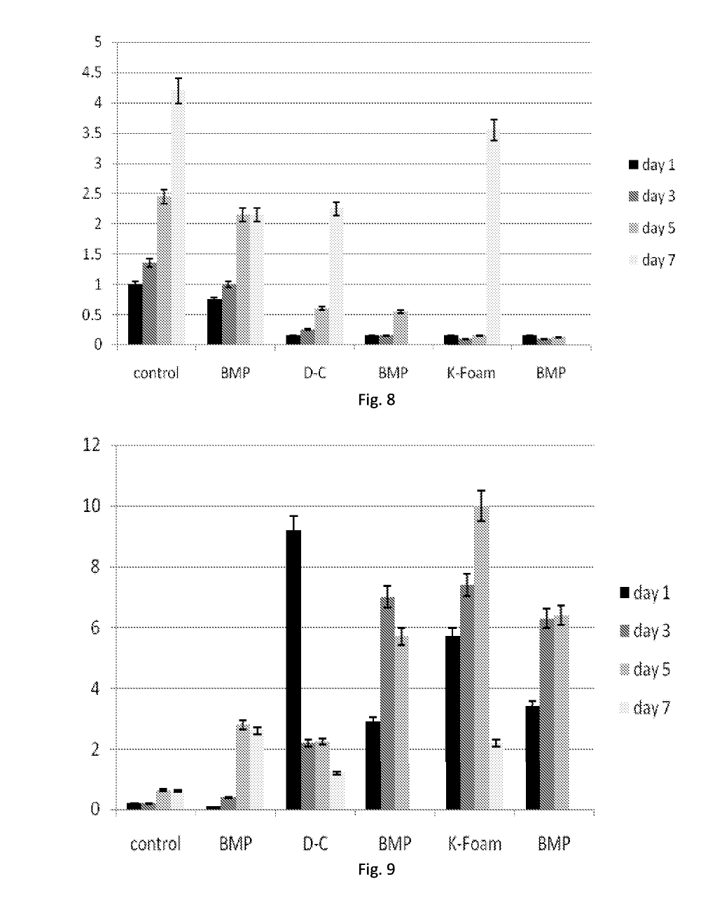

Replication of C2C12 cells was determined using a standard MTT assay that measures cell respiration. Relative numbers of cells among treatment wells can thereby be fairly accurately determined. In these studies cell number was found to increase with increasing time in culture, as anticipated. At any given time point the number of cells in the TCP wells exceeded the numbers in culture of cells applied to either carbon foam (FIG. 8). Significantly, perhaps, a lag in cell number in the carbon foam cultures was seen but not in the TCP wells. Consistent with the literature of differentiating pluripotent stem cells, BMP at a concentration of 200 ng/ml reduced cell replication below untreated controls for all treatments. BMP+foam exposure proved to be cytotoxic since cell numbers approached 0 by day 7 by the criteria imposed by the MTT assay.

Alkaline phosphatase is a key enzyme induced early in the cycle of events leading to osteoblast differentiation. Levels of this enzyme activity are close to 0 in unstimulated C2C12 cells, with induction by BMP occurring rapidly, within a day or two of cytokine exposure. Consistent with this dogma AlkPhos was indeed provoked by BMP in control cell cultures between days 3 and 5 (FIG. 9). Interestingly, AlkPhos activity in cells exposed to both carbon foams in the absence of BMP increased circa 5- to 50-fold by day 3. For DUOCEL® vitreous carbon an additional 2-3-fold increment of activity was provoked by BMP. This cytokine-synergistic effect was not observed for graphitic KFOAM®. Consistent with the MTT viability results, AlkPhos activity was undetectable in day 7 cultures where cell numbers had declined in the MTT assay (compare FIG. 8).

### 5. Conclusions

We were somewhat surprised by the lack of BMP elution detected from rinsed, preloaded carbon discs since protein binding by several forms of carbon materials is strong (but not covalent) and can be irreversible. It is likely that the tightly bound BMP retains biological activity based upon the alkaline phosphatase induction results. The C2C12 cells tested exhibited relatively good biocompatibility in contact with the vitreous and graphitic foams. Replication, viability and inductivity were maintained for at least 5 days in culture. The decrement in these parameters at day 7 are likely due to poor material penetration of nutrients and/or oxygen which owing to neovascularization seen in these materials in previous animal studies, should not be a problem in vivo. We were somewhat surprised to see osteoinductivity exhibited by the foams in the absence of BMP. This important property has been observed in other non-biologic implant materials as well. This property, in combination with positive synergy seen with BMP-2 may well prove to be decisive for providing a novel bone bridging, void filling material for the orthopedic community.

### Example 3

### Evaluation of Osteoblast Integration into Porous Carbon Foam Matrix

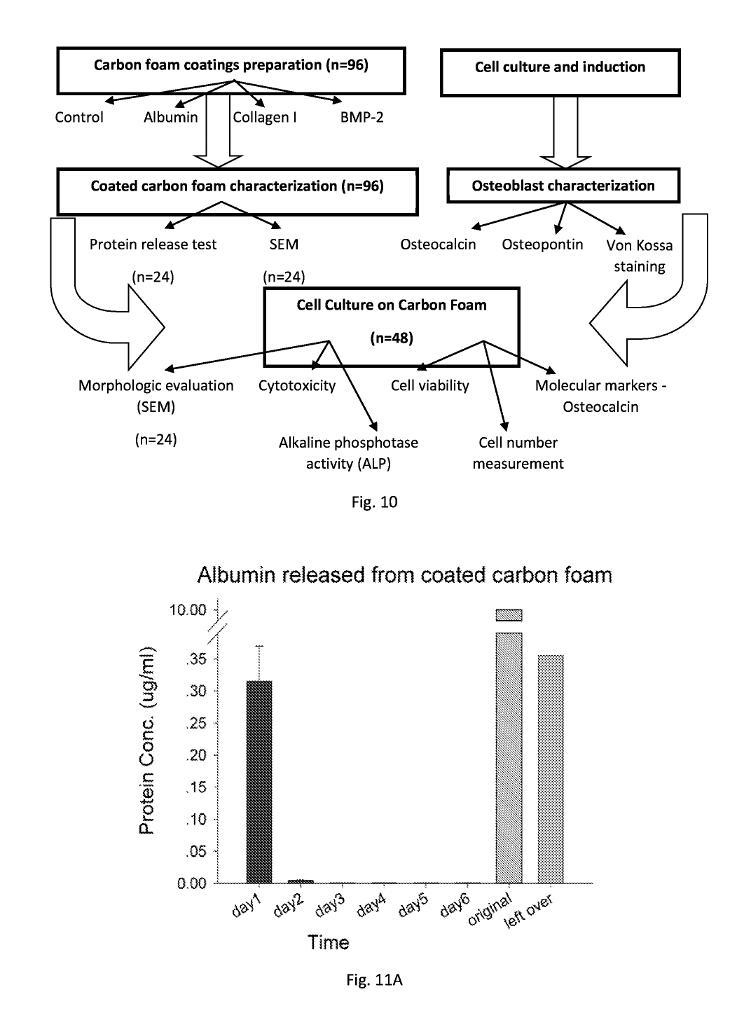

The objective of this study was to examine several factors associated with cell attachment to the surface (osteoblast integration) of the carbon foam and study the effects of various coatings applied to the carbon foam scaffold. A flowchart of the study protocol is shown in FIG. 10.

### 1. Materials and Methods

Reticulated vitreous carbon foam (DUOCEL® RVC carbon foam; ERG Materials and Aerospace Corporation, Oakland, Calif.) with 80 PPI, 3% relative density and pore sizes ranging from 40-250 μm was used for these experiments. Four different types of carbon foam conditions were examined: (A) Original (uncoated) carbon foam; (B) Albumin-coated carbon foam; (C) Collagen type I-coated carbon foam; and (D) Bone Morphogenetic Protein 2 (“BMP-2”) coated carbon foam. The original (uncoated) carbon foam sample group was selected as the baseline for comparison of the other sample groups. A total of ninety six discs (24 for each sample group) were machined and cut into 1-cm diameter and 2-mm thick cylinders. The discs were cleaned with alcohol to remove residual carbon dust from machining.

### 2. Carbon Foam Coatings Preparation

The discs were subjected to one of 3 pretreatments: (1) immersion in 10 μg/ml human serum albumin (HSA) with distilled water for 24 hours at 37° C.; (2) immersion in collagen type I solution for 24 hours at 37° C.; or (3) immersion in 10 μg/ml BMP-2 with distilled water for 24 hours at 37° C. Carbon foam discs immersed in distilled water served as a control. A low pressure vacuum system was used to facilitate the absorption of the coatings into the pores inside the carbon foam discs. The treated carbon foams were then carefully centrifuged to remove excessive solution. All samples were then air-dried in a biology hood overnight. Residual pretreatment solutions were collected and measured for albumin, collagen I, or BMP-2 concentration using an enzyme-linked immunosorbent assay (ELISA).

### 3. Coated Carbon Foam Characterization

a. In Vitro Protein Release Test:

Coated carbon foam discs (6 samples/group) were placed in a 24-well plate with 1 ml of phosphate buffer solution (PBS) with pH 7.4 release buffer. The carbon foam discs were then incubated at 37° C., the release buffer was collected and replaced with fresh buffer daily for 6 days. All samples were then stored at −20° C. until analysis. The albumin, collagen type I or BMP-2 released from the samples was then assayed using ELISA. Duplicate series of 8 two-fold dilutions of each coated material ranging from 1,000 to 4 ng/ml were prepared, and this served as a standard data set to determine protein concentrations of the sample groups. The absorbance of the samples and standards were measured at 450 nm wavelength using a microtiter plate reader, and the cumulative release of albumin, collagen type I or BMP-2 was extrapolated from the standard curves.

b. Scanning Electron Microscopy (SEM)

All the samples were mounted onto SEM stubs, and the porous properties of the carbon foam (SEM images) were observed and evaluated at 10 kV or 15 kV using a scanning electron microscope.

### 4. Cell Culture and Induction

Bone marrow cells (BMC) were obtained from the bone marrow of female Lewis rats. Following euthanasia by CO2 asphyxiation, femora were aseptically excised, the metaphyseal ends were cut off and the marrow was flushed from the medullary cavity with 10 ml of Dulbecco's Modified Eagle medium (DMEM) using a syringe with a 22-gauge needle. Cell clumps were dispersed by repeatedly pipetting the cell suspension, and low-density bone marrow mononuclear cells isolated using density centrifugation over Histopaque®-1083. Cells were then washed with PBS, and prepared for culture and differentiation of osteoblasts. BMCs were induced to differentiate into osteoblasts in complete media consisting of DMEM supplemented with 10% fetal bovine serum (FBS), 10 mM β-glycerol phosphate, 100 μM L-ascorbic acid, and 10 nM dexamethasone, 2 mM glutamine, 100 U/ml penicillin, 100 ug/ml streptomycin, and 10-4 M L-ascorbic acid. The induced BMCs were then seeded on the coated carbon foam discs and cultured in an incubator at 37° C. for seven days.

### 5. Osteoblast Characterization

a. Osteocalcin, Osteopontin, and Collagen Type I

On the second passage, the induced osteoblasts were fixed in 4% paraformaldehyde, permeated with 0.01% Triton X-100 in PBS, and incubated in 1% block serum for 1 hour at 37° C. The cells were then incubated with anti-osteocalcin, osteopontin, or collagen type I for 1 hour, and visualized using Alexa Fluor conjugated (Molecular Probes, Eugene, Oreg.) (for osteocalcin and osteopontin) or Alexa Fluor 488-conjugated (Molecular Probes, Eugene, Oreg.) (for collagen type I) secondary antibody. The cells were examined under a fluorescence microscope. Nuclei were counterstained with DAPI (Molecular Probes, Eugene, Oreg.).

b. Von Kossa Staining

The presence of calcium deposits was demonstrated by von Kossa staining. Potential osteoblasts were rinsed in PBS and fixed in 4% paraformaldehyde for 30 minutes, and then incubated with 1% silver nitrate solution (Sigma-Aldrich, US) under ultraviolet light for 20 minutes. Un-reacted silver was removed by 5% sodium thiosulfate (Sigma-Aldrich, US) for 5 minutes. The stained slides were then observed under microscope after permanent mounting.

### 6. Cell Culture on Carbon Foam

Induced osteoblasts in 50 μl suspensions (3.5×10⁶ cells/ml) were respectively loaded onto each scaffold in 24-well plates. The scaffolds were left undisturbed in a 37° C. incubator for 2 hours to allow cells to attach to the scaffold, after which the seeded cells on materials were kept in culture using the same osteogenic media. Medium was changed every 3 days, and at Day 7 samples were harvested for morphological and biochemical evaluation. Culture dish cells were used as the control.

### 7. Morphological Evaluation

To prepare for SEM examination, membranes with Day 7 culture cells were fixed with 2% glutaraldehyde for 60 minutes at 4° C. The samples were then washed twice in PBS and exposed to 2% osmium tetroxide for 60 minutes. Once the samples had been rinsed in distilled water, they were dehydrated through a graded series of ethanol (20%, 40%, 60%, 80%, 95%, and 100%) for 5 minutes. The dehydration process was completed in hexamethyl disilazane (HMDS) for 10 minutes. After air-drying, the samples were spot-coated with gold and mounted onto SEM stubs. SEM images were observed at 10 kV or 15 kV, and saved for the morphology evaluation of the induced osteogenic cells on the scaffolds.

### 8. Cytotoxicity

Cytotoxicity of carbon foams was quantitatively assessed by the measurement of lactate dehydrogenase (LDH) leakage. A CytoTox 96® assay was used to measure the ratio of lifeless cell to live cells on the bone or composite scaffold. LDH is an outstanding indicator of cell death and damage. Lifeless cells release LDH during culturing, therefore, it is essential to collect the cultured medium along with the lysis buffer supernatant. 10 μl of aliquots of the medium was mixed with 200 μl LDH reagent, and the cultured medium and lysis buffer solutions for each of the four groups were transferred to their corresponding well. A spectrophotometer (Spectra MAX Gemini XS) was used to measure the absorbance of solutions at 490 nm wavelength. Higher readings correspond to a higher toxic environment for osteogenic cells.

### 9. Cell Viability

Cell viability was assayed by analyzing the mitochondrial activities in the cultured cells on the discs. The alamarBlue® assay (BioSource, US) was used to determine the activity of the cells after 7 days of cell culture. AlamarBlue® reagent is a valuable tool used to ensure cell proliferation on the discs through correlating cell number to the absorbance values. In contrast to cytotoxicity tests, the cultured medium was removed completely with PBS and fresh medium was added to cover the composite samples. 3 ml of new conditioned media supplemented with 200 μl of alamarBlue® was added to each well, and incubation was continued at 37° C. with 5% CO2 for 4 hours. The culture medium was then transferred to a 96-well plate and read on a spectrophotometer (Spectra MAX Gemini XS) at excitation wavelength 570 nm, emission wavelength 600 nm, and data was collected and analyzed using SoftMax PRO. The alamarBlue® absorbance of DNA values were calculated for each sample.

### 10. Cell Number Measurement

Cell numbers were determined by a fluorometric quantification of DNA on the carbon foam construct. After the alamarBlue® assay, the cell-scaffolds were rinsed with PBS, followed by 1 ml lysis buffer and 2 minutes ultrasonic. The lysate was then saved into a specific tube. 100 μl of supernatant sample were mixed in 1.5 ml of 200 ng/ml Hoechst 33258 fluorescent dye (Sigma-Aldrich, US) and read at EX 350 nm and EM 455 nm by fluorometer. The DNA concentration in the samples was determined against a DNA standard curve.

### 11. Alkaline Phosphatase (ALP) Activity

ALP activity by osteogenic cells was measured using a spectrophotometer. After the previous freeze-thaw cycle (freeze at −80° C. for 30 minutes, and thaw at 37° C. for 30 minutes) and homogenization for the DNA assay, 100 μl of the sample was removed from the lysate to which 100 μl of p-nitrophenyl phosphate solution was added. After 30 minutes incubation at 37° C., the production of p-nitrophenol in the presence of ALP was measured at an absorbance of 405 nm wavelength. The measurement of the ALP assay was normalized against the amount of total DNA in each sample.

### 12. Molecular Markers (Osteocalcin)

Cell lysates were used for the assay of osteocalcin using a sandwich ELISA. 2 μg/ml of primary antibody was coated and incubated overnight at 4° C. The plates were washed 3 times with PBS, dispensed with 200 μl of 5% milk and incubated at 37° C. for 4-6 hours. Samples with 50 μl of supernatant were added to the coating plate and incubated overnight at 4° C., followed by washing the plate and adding 100 μl of 1 μg/ml antibody, followed by incubating at 37° C. for 1 hour. Plates were washed and then 20 μl of streptavidin was added into 10 ml of PBS, and incubated at 40° C. Next, 2 pNPP was dissolved per 10 ml of diethanolamine buffer, followed by incubation at 37° C. in the dark for 5-20 minutes. A spectrophotometer (Spectra MAX Gemini XS) was used to measure the absorbance of solutions at 405 nm wavelength, and the absorbance of ELISA was normalized against the amount of total DNA in each sample.

### 13. Results

a. Characterization of Coated Carbon Foam

(1) Protein Release Test

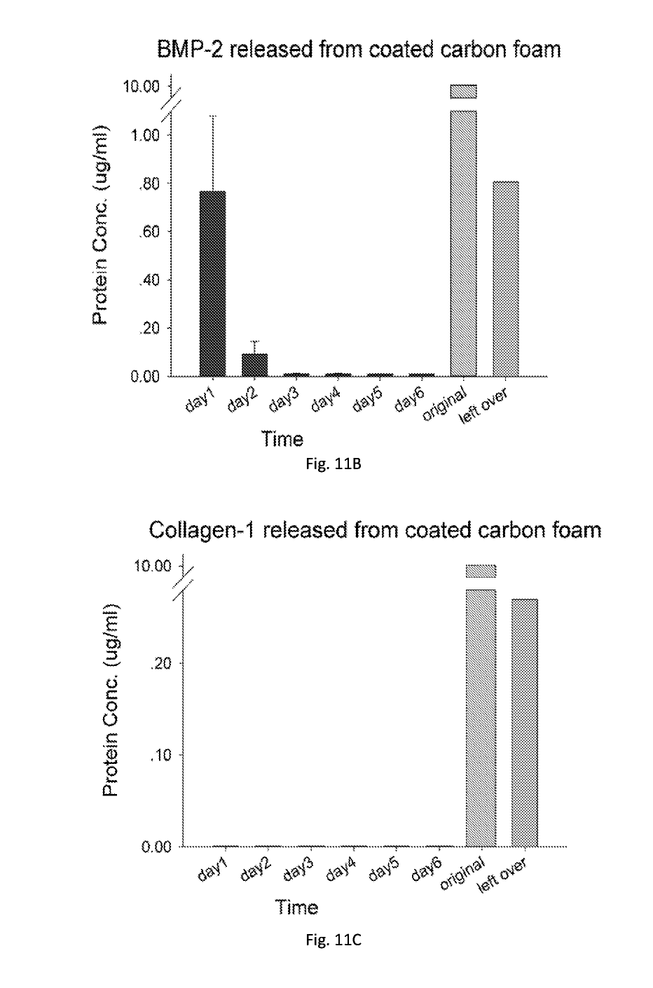

Both albumin and BMP-2 exhibited a minimal burst release in the first 24 hours with the albumin and BMP-2 releasing 0.108% and 0.2733%, respectively (FIG. 11A-B). These burst releases were followed by significantly reduced release rates thereafter. Collagen type I did not exhibit the same burst release profile as seen with albumin and BMP-2 in the first 24 hours (FIG. 11C). Collagen type I was released steadily at the rate of 0.0003% during 6 days of assessment. This negligible release of collagen type I from the carbon foam allowed a high retention of protein on the surface of the biomaterial, which subsequently contributed to its biological effect on attached cells.

While the cumulative releases of albumin and BMP-2 from carbon were more than that of the collagen type I, the retentions of albumin and BMP-2 still were up to 99.89% and 99.68%, respectively. Therefore, it is possible that the minimal release rates have allowed sufficient amounts of albumin and BMP-2 to remain, and then sustain cell adhesion, proliferation, and the formation of bony matrix afterward. The stable and long retention of protein on the scaffold will allow for the increase of release duration and the local protein concentration. This pattern of release may rarely lead to a distant spread of the protein, and subsequent ectopic bone formation.

TABLE II
Protein release from coated carbon foam

| | Albumin | | BMP-2 | | Collagen type I | |
|---|---|---|---|---|---|---|
| | Concentration | Rate | Concentration | Rate | Concentration | Rate |
| | (μg/ml) | (%) | (μg/ml) | (%) | (μg/ml) | (%) |
| Day 1 | 0.31510 ± 0.05510 | 0.1081 | 0.76700 ± 0.31370 | 0.2733 | 0.00074 ± 0.00002 | 0.0003 |
| Day 2 | 0.00442 ± 0.00122 | 0.0015 | 0.09230 ± 0.05300 | 0.0329 | 0.00074 ± 0.00005 | 0.0003 |
| Day 3 | 0.00061 ± 0.00007 | 0.0002 | 0.01080 ± 0.00105 | 0.0038 | 0.00071 ± 0.00001 | 0.0002 |
| Day 4 | 0.00084 ± 0.00008 | 0.0003 | 0.01100 ± 0.00118 | 0.0039 | 0.00069 ± 0.00002 | 0.0002 |
| Day 5 | 0.00070 ± 0.00002 | 0.0002 | 0.01020 ± 0.00082 | 0.0036 | 0.00069 ± 0.00003 | 0.0002 |
| Day 6 | 0.00076 ± 0.00010 | 0.0003 | 0.00957 ± 0.00025 | 0.0034 | 0.00072 ± 0.00004 | 0.0002 |

| | | | |
|---|---|---|---|
| Original | 10.00000 | 10.00000 | 10.00000 |
| Left over | 0.35570 | 0.80780 | 0.26970 |

The percent release of protein at each time point was determined as follows:

$$Release Rate = \frac{amount of protein released at day X}{( 50 ug protein - amount of protein left over ) / 6 samples} \times 100 \%$$

b. Osteoblast Characterization

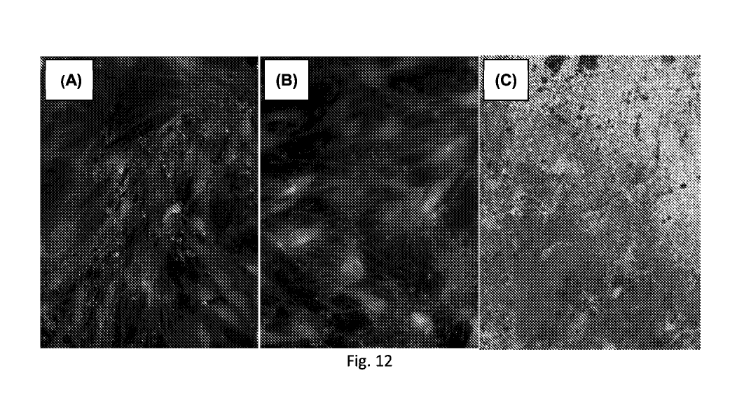

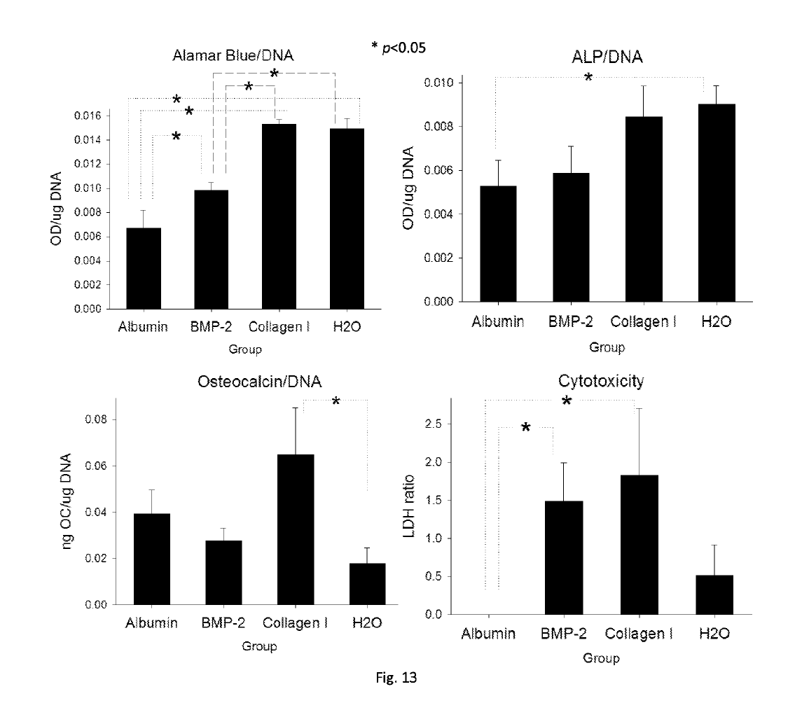

The capacity of the induced osteoblasts to express osteocalcin, osteopontin, and collagen type I was examined by immunocytofluorescence (FIG. 12). While the expression of osteocalcin (in red, FIG. 12A) and osteopontin (in red, FIG. 12B) was prominent in the induced osteoblasts, these cells were further identified by positive staining for collagen type I (in green, FIG. 12A-B), indicating that the induced cells possessed the distinguishable osteoblastic phenotype. To demonstrate the ability of cells to mineralize the matrix, cells cultured on Petri dishes were subjected to von Kossa staining to reveal calcium deposition (FIG. 12C). The darkly stained mineralized nodules were visualized by silver nitrate, indicating normal osteoblast function in conditioned culture.

TABLE III
Cell culture on carbon foam

| | alamarBlue ® | ALP | Osteocalcin | |
|---|---|---|---|---|
| | (OD/ug DNA) | (OD/ug DNA) | (ng/ug DNA) | LDH |
| Albumin | 0.007 ± 0.003 | 0.005 ± 0.003 | 0.039 ± 0.023 | −0.951 ± 1.041 |
| BMP-2 | 0.010 ± 0.001 | 0.006 ± 0.002 | 0.028 ± 0.011 | 1.491 ± 1.126 |
| Collagen I | 0.015 ± 0.001 | 0.008 ± 0.003 | 0.065 ± 0.045 | 1.829 ± 1.961 |
| H2O | 0.015 ± 0.002 | 0.009 ± 0.002 | 0.018 ± 0.013 | 0.520 ± 0.786 |

To test the effects of different protein coatings on osteoblasts, we analyzed cell viability, ALP activity, osteocalcin expression, and cell damage of osteoblasts using spectrophotometry or spectrofluorometry at the single-cell level by culturing cells on carbon foam in vitro for 1 week. Relative quantification of activities at the single-cell level was employed in this study, because augmented cellular functions and viabilities should definitely be pursued when cell amounts could be controlled relatively easily as assembling engineered bone. Comparisons were made among the groups of albumin, BMP-2, collagen type I, and H2O treated carbon foams (FIG. 13).

Collagen type I and H2O treated carbon foams led to higher cell viability than the albumin and BMP-2 groups, which were represented by the alamarBlue® reading normalized by DNA amount in each group. ALP activity and osteocalcin expression are functional and maturational indicators of the osteoblast phenotype, and partially correlated with capacity of bone formation. It seemed that the ALP activity responded to varying materials in a similar way to cell viability, with the higher values for ALP observed at the groups of collagen type I and H2O. Compared with the other groups, the collagen type I stimulated higher osteocalcin expression, although the statistical significances were obscure with the exception of collagen type I vs. H2O. BMP-2, the well-known osteogenic protein which has been widely used in bone reconstructive surgery, was expected to facilitate the osteoblast differentiation and function. In the current study, however, BMP-2 appeared not to provide a more favorable environment for osteoblast function, which was observed as the lower production of ALP and osteocalcin. Paradoxically, collagen type I and BMP-2 treated carbon foam led to an apparent cytotoxicity, which was indicated by a higher reading of the LDH ratio. It is difficult to interpret the concurrent promoting and inhibiting effect of collagen type I on the osteoblasts, which were evidenced by the higher readings in the alamarBlue® and LDH assays. The albumin group, however, exhibited the lowest cell viability (i.e., alamarBlue®/DNA) as well as the lowest adverse effect on the cells (i.e., LDH). In contrast, H2O-treated carbon foam, with the absence of any protein coating, demonstrated promising properties: higher cell viability, lower cell toxicity, and higher ALP activity, with the exception of the lower osteocalcin production.

### Example 4

### Evaluation of Particulate Carbon Foam

Work completed here demonstrates that carbon foam particulates share with its monolithic carbon foam parent material the ability to both bind BMP-2 and to promote the attachment of osteoblast and stem cells to the material surface. These properties contribute to a graft material that is osteogenic, osteoinductive as well as osteoconductive. These three foundational attributes, in combination with the generated “porosity” and “pore geometry” of aggregated particulates that mirrors to a greater or lesser degree the geometry of the parent foam material when the particulates are constrained in a carrier matrix give rise to implant materials that promote significant de novo bone formation at surgically-created defect sites. The proof-of-concept was developed in part using an in vitro cell-based osteopromotive assays, high throughput ectopic bone formation and clinically-mimetic critical bone defect animal models.

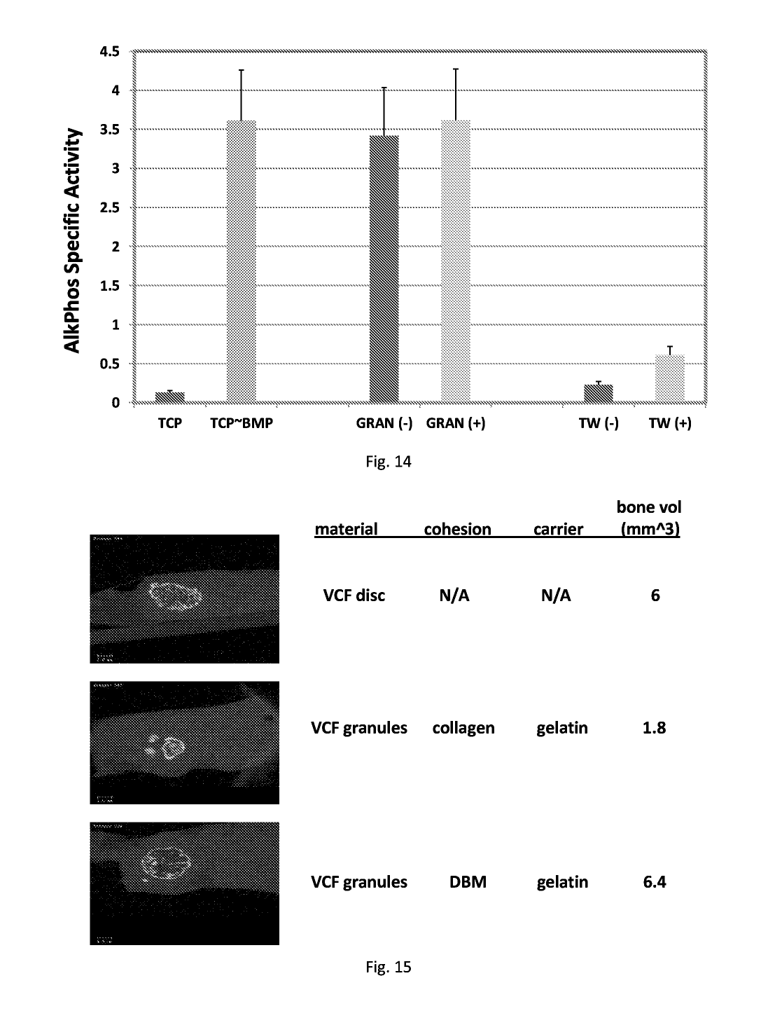

FIG. 14 is a graph demonstrating BMP-2 binding to carbon foam particulates using alkaline phosphatase induction in C2C12 mouse myoblast cells. Replicate lots of 10 mg carbon foam particulates were immersed in 500 ng BMP-2 in 200 ul PBS. After an 18 hr soak, carbon foam particulates from 3 replicates were separated from the BMP solution (GRAN(−)) and placed in contact with C2C12 cells growing on a plastic surface. Three other carbon foam particulate replicates were flash-frozen in the BMP-2 soak solution (GRAN(+)) and likewise placed in contact with growing cells. Replicates of these same groups were placed into transwells that allowed contact with the tissue culture medium but prevented direct cell contact (TW(−) and TW(+)). As a control 500 ng BMP-2 was added directly to the medium in which C2C12 cells were growing. Five days following the addition of granule-bound or free BMP the cells were lysed and alkaline phosphatase specific activity (a measure of osteogenic induction) was measured (+/−SEM).

The data in FIG. 14 show that the amount of alkaline phosphatase activity induced in C2C12 cells by BMP-2 coating carbon foam particulates (aka “VCF granules”) was equivalent to that induced by soluble BMP-2 added to the culture medium (compare TCP˜BMP with GRAN (−)). Significantly the alkaline phosphatase induced by carbon foam particulates soaked in BMP-2 solution and then lyophilized (thus delivering the total BMP-2 dose to the cells) was not increased over the control or soak-minus replicates. This outcome indicates that BMP is rapidly and quantitatively bound by carbon foam particulate surfaces and further, that biological activity is retained during and subsequent to the binding step. Of equal importance is the finding that neither the coated/segregated nor coated/lyophilized materials in transwells (TW(+) and TW(−)) were capable of inducing alkaline phosphatase illustrating the excellent retentive properties of the material. This unexpected property of the carbon foam particulates gives rise to a “slow” elution profile that translates into exceptionally good new bone formation when such composites are implanted into animals (see below). Such an elution profile has been measured on numerous occasions under many experimental conditions by the applicants supporting the often cited contention in the literature that a small burst followed by slow release over several days is optimal for therapeutic BMP-2 delivery.

A high throughput ectopic bone formation animal model in which graft materials are implanted into a bleeding muscular site was also developed to support the efficacy of carbon foam (aka vitreous carbon foam or VCF) particles and composites thereof. FIG. 15 shows images of ectopic bone formation induced by carbon foam particulates or carbon foam particulates coated with BMP-2 implanted into an abdominal muscle pouch of a Lewis rat for 30 days as measured using microCT. The amount of new bone formation induced by a control implant (VCF 3D disc+1000 ng BMP-2) was compared with BMP-2-coated carbon foam particulates composited with either collagen+gelatin or DBM+gelatin. Two-dimensional uCT images are shown at left and measured bone volumes (mm³) tabulated at the right (mean of 3 measurements). As shown in FIG. 14, carbon foam particulates coated with BMP-2 and then composited with either collagen/gelatin matrix or DBM/gelatin matrix compared favorably with carbon foam particulates (3D VCF) discs coated with BMP-2 as measured by uCT to derive new bone volume. These results have been replicated with many of the VCF granule/composite formulations depicted in Table IV (below).

The following input criteria were applied to the development of VCF granules for grafting applications:

1. Identification of polyethylene terephthalate (PET) mesh as a suitable biocompatible containment material for carbon foam particulate composites containing osteopromotive or therapeutic coatings alone (without carrier). This configuration is permissive of through-growth of new bone while at the same time providing defect-site conformation and ease of surgical application.

2. Inclusion of a carrier material that contributes to the cohesiveness of the bulk material as a result of its solubility in the liquid phase. This property of the composite gives rise to the ability to deliver the bulk graft material as either a hand-molded object or extruded from a syringe (with or without percutaneous, syringe injection, capability).

3. Inclusion of an insoluble material contributing to the coherence of the composite through increasing the viscosity and providing a level of surface tension sufficient to reduce dissolution of the carrier component in contact with bodily fluids at the graft site (through reducing the effective water concentration). A shredded collagen product capable of forming a hydrogel has been found to perform this role well.

4. Optimization of BMP-2 cytokine adhesion to the VCF particles prior to merging with handling components to provide an osteoinductive stimulus.

5. It was also recognized that dilution the BMP-2/VCF granule component could result in unacceptable dilution of osteoinductivity and in response to this concern VCF-BMP-2 embodiments were developed and tested in which the coated granules alone have been incorporated into fibrin gels or enveloped in PET mesh material culminating in bone graft substitutes that have lost the ability to be delivered by syringe ejection but that can be delivered using either a large-bored applicator or placed manually into irregularly-shaped bone voids.

From the aforementioned input criteria a simple test matrix was developed. This consisted of developing pilot formulations of carbon foam particulates (aka “VCF granules”), carrier and cohesive material that allowed for adequate representation of VCF (and, thus, bound BMP-2 cytokine) in the final product to afford adequate bone formation. This decision was based upon previous in vitro and animal studies in which the induction of bone cell-forming features were identified. The formulations noted in Table IV were prepared in a sterile tube, back-loaded into 3 cc syringes, and the material extruded onto a clean Parafilm surface. Immediately following extrusion, the bead of composite material was immersed in 37° C. phosphate buffered saline (PBS) and observed for 15 minutes to identify any tendency towards “dissolution.” This terminology was applied to describe the outflow of visible solid materials away from the body of the bead as a result of solubilization and outward diffusion of the carrier material. The dissolution-tested materials were then held at 37° C. (to mimic body temperature) and “irrigation-resistance” was monitored as the propensity of the warmed material to resist becoming incoherent (as a result of phase transition from gel to liquid) during agitation on a rocking platform at 37° C.

TABLE IV
Composite graft formulations tested in dissolution and irrigation-resistance assays.
The actual formulations were comprised of 5X the volumes/masses noted.

| | VCF | Cohesion | Carrier | PBS | Dissolution | Irrigation-R |
|---|---|---|---|---|---|---|
| key | (mg) | (mg) | (mg) | (ml) | (time @ 25° C.) | (time @ 37° C.) |
| A | 60 | shredded collagen | N/A | N/A | good | good |
| | | (160) | | | (30 min) | (30 min) |
| B | 60 | Shredded collagen | Gelatin (100 mg) | 0.3 | Excellent | Excellent |
| | | (160) | | | (>60 min) | (35 min) |
| C | 60 | Shredded collagen | Carboxy methyl | 1.3 | v. good | v. good |
| | | (160) | Cellulose (120) | | (40 min) | (25 min) |
| D | 60 | N/A | Gelatin −180 | 2.3 | Excellent | Excellent |
| | | | | | (>60 min) | (35 min) |
| E | 60 | N/A | Carboxy methyl | 3.3 | v. good | Good |
| | | | Cellulose (120) | | (30 min) | (15 min) |
| F | 60 | DBM (140) | Gelatin (180) | 0.6 | Excellent | Excellent |
| | | | | | (>60 min) | (35 min) |
| G | 60 | DBM (140) | Carboxymethyl | 0.6 | Excellent | Excellent |
| | | | Cellulose (120) | | (>60 min) | (35 min) |
| H | 60 | DBM (140) | Glycerol (450) | 0.3 | Poor | Poor |
| | | | | | (5 min) | (1 min) |
| I | 60 | DBM (140) | CaSO4 (180) | 0.45 | Fair | Poor |
| | | | | | (10 min) | (5 min) |
| J | 60 | DBM (140) | Phosphatidyl | 0.6 | Fair | Poor |
| | | | Choline (400) | | (10 min) | (5 min) |
| K | 60 | DBM (140) | Hyaluronic | 0.6 | Fair | Poor |
| | | | Acid (400) | | (10 min) | (5 min) |

### RESULTS AND CONCLUSIONS

All of the material combinations tested (as distinguished chiefly by carrier type) performed acceptably based upon the expectation of syringe ejection and moldability (not quantified here) and survival of coherence upon immersion in 37° C. PBS (dissolution). Several of the carrier-graft material combinations performed poorly due to lack of irrigation-resistance including glycerol, CaSO4, phosphatidylcholine, and hyaluronic acid. The carriers, gelatin and carboxymethyl cellulose, performed well by all criteria and will be considered as preferred embodiments in this prospectus. Evaluation and testing of additional materials is ongoing. Both gelatin and carboxymethyl cellulose carriers will undergo further testing in animal models to ascertain their relative abilities to support new bone formation in more challenging circumstances.

The particulate VCF particulate grafts described herein were developed to address shortcomings in the art. In this context we note the following performance issues addressed by the materials described:

1. Exceptional BMP-2 (and other clinically-relevant) cytokine adsorption and retention at the graft site. This outcome is generally acknowledged to increase the titer of repair cells at the defect site (through graft-site restriction of cytokine) as well as through the reduction of cytokine dosage which mitigates untoward off-target effects. We note that these performance attributes will contribute to the repertoire of FDA-approved clinical applications for currently approved therapeutics.

2. Improved handling and adaptability to defect site. That is, synthetic bone graft substitutes are relatively brittle and not conducive to conforming to irregular defects due to their chemical compositions and dimensions. The same is true of the allograft and autograft bone graft materials harvested from donors or patients, respectively. Significantly, the current bone graft substitute standard of care (BMP-2 applied to a collagen sponge) experiences compressive forces during handling and implantation that result in ejection of cytokine solution. This results in both reduced local efficacy and undesirable off site ectopic bone formation.

3. The VCF particulate graft materials also outperform other types of graft materials owing to geometry. That is, application to the wound site of a scaffold having an interconnected generated “porosity” that is optimal both dimensionally and in terms of pore diameter. Other graft materials (most notably glasses, ceramics and non-vitreous carbon chemistries) fail to provide this generally-accepted narrow range of porosities represented here through iterative experimental discovery on the part of the applicants. The significant outcome of these optimizations is rapid and profuse development of a vascularized tissue bed which is generally acknowledged to be a fundamental prerequisite for successful grafting procedures.

Advantageous and non-obvious features of the invention include:

1. That BMP-2 (and other relevant protein therapeutics) binding to the carbon foam particulates is achieved through non-covalent bonding interactions and therefore resists dissolution of hydrodynamic elution. Grafts prepared using the materials and methods described herein are not simply “therapeutic-eluting” (as is true of synthetic polymers, glasses, ceramics, metals and other commonly-used graft materials) but are “therapeutic-retentive.”

2. The materials and composites described herein are not only permissive of excellent cell binding, replication and therapeutic-specific differentiation but serve to guide early neo-vascularization.

3. The materials described herein, when composited with the composites suggested, are slowly degraded through cellular interaction thereby providing a resorbable graft.

4. Unlike solid carbon foam constructs described in earlier publications and patent applications the carbon foam particulates and composites can be molded, injected, and extruded to fit irregular defect shapes and volumes thereby providing easy surgical implantation while expanding clinical application(s).

This invention was designed with improvement of patient care in mind. The patient population will benefit by/from the introduction of this technology through increased surgical options for regenerative surgical procedures, favorable clinical outcomes with fewer revision surgeries, and reduced out-of-pocket expenses. These outcomes are achieved via the design of one or more of the posited embodiments that incorporate efficient delivery/retention of therapeutic agents, options for the application of the appropriate graft (retained in a mesh, syringe or percutaneous delivery), and conforming the graft material to the wound/defect site.

These materials have undergone intensive scrutiny in both in vitro and in vivo animal model testing modes. The results of these studies have revealed that particulate vitreous carbon foam-derived particles are biocompatible, permissive of cell attachment and differentiation, bind regeneratively-important growth factors in an active elutable form, and provide a 3-dimensional substrate that is permissive of neo-vascularization. The latter outcome is critically important for the initiation of healing of all tissue defects including musculoskeletal, cardiac, hepatic, vascular and epidermal.

1. A composition for tissue and/or bone regeneration, said composition comprising a plurality of carbonaceous particulates and at least one osteopromotive and/or therapeutic agent.

2. The composition of claim 1, said carbonaceous particulates having a maximum surface-to-surface dimension of less than about 1,000 microns.

3. The composition of claim 1, wherein said carbonaceous particulates are non-macroporous granular bodies derived from carbon foam.

4. The composition of claim 1, wherein said carbonaceous particulates are powdered carbon foam.

5. The composition of claim 1, wherein said composition is a free flowing dry composition.

6. The composition of claim 1, wherein said carbonaceous particulates are physically mixed with said at least one osteopromotive and/or therapeutic agent.

7. The composition of claim 1, wherein said carbonaceous particulates comprise a coating of said at least one osteopromotive and/or therapeutic agent.

8.-9. (canceled)

10. The composition of claim 1, further comprising a carrier matrix.

11.-12. (canceled)

13. The composition of claim 10, wherein said composition is a flowable composite in the form of a self-sustaining body.

14. The composition of claim 10, wherein said carrier is selected from the group consisting of gelatin, carboxymethyl cellulose, hyaluronic acid, phosphatidyl choline, atelocollagen, and combinations thereof.

15. The composition of claim 10, wherein said carrier matrix is a curable polymer.

16. The composition of claim 1, further comprising a cohesion agent selected from the group consisting of shredded collagen, demineralized bone matrix, non-crosslinked collagen fibers, chitin, chitosan, silk, carbon fibers, and combinations thereof.

17.-18. (canceled)

19. An implant for tissue and/or bone regeneration, said implant comprising a composition according to claim 1; and a biocompatible mesh container, said composition being enclosed within said container.

20. An implant for tissue and/or bone regeneration, said implant comprising a composition according to claim 1; and a biocompatible scaffold, said composition being embedded within said scaffold.

21. (canceled)

22. A method for tissue and/or bone regeneration in a subject having a need therefor, said method comprising:
(a) providing a composition according to claim 1; and
(b) implanting said composition into said subject at an implantation site having a bone defect or tissue damage.

23. The method of claim 22, wherein said bone defect is selected from the group consisting of calvarial defects, osteochondral defects, fractured vertebrae, intervertebral or posterolateral spinal fusion, alveolar ridge augmentation, extraction socket restoration, illiac crest defect repair.

24. The method of claim 22, wherein said implanting comprises molding said composition to fit said bone defect to provide a precise interface with undamaged bone adjacent to the bone defect.

25. The method of claim 22, wherein said providing (a) comprises providing a syringe having a reservoir loaded with said composition, said implanting (b) comprises dispensing said composition from said syringe at the site of implantation.

26. A kit for tissue and/or bone regeneration in a subject having a need therefor, said kit comprising:
a composition according to claim 1; and
instructions for implantation thereof in said subject at an implantation site having a bone defect or tissue damage.

27. The kit of claim 26, said kit further comprising a syringe having a reservoir loaded with said composition for dispensing into said site of implantation.

28.-29. (canceled)
